(19) United States
(12) Patent Application Publication
(10) Pub. No.: US 2009/0264470 A1
(43) Pub. Date: Oct. 22, 2009

(54) CB1 Antagonists and Inverse Agonists

(76) Inventors: Teresa A. Bennett, Malaga (ES); James R. Hauske, La Jolla, CA (US); Fu-Yue Zeng, Lakeland, TN (US)

Correspondence Address: ROPES & GRAY LLP, PATENT DOCKETING 39/41, ONE INTERNATIONAL PLACE, BOSTON, MA, 02110-2624, US

(21) Appl. No.: 12/224,189

(22) PCT Filed: Feb. 21, 2007
(86) PCT No.: PCT/US2007/004681

§ 371 Date: Dec. 15, 2008

Related U.S. Application Data
(60) Provisional application No. 60/775,493, filed on Feb. 21, 2006.
(60) Provisional application No. 60/790,064, filed on Apr. 6, 2006.
(60) Provisional application No. 60/817,835, filed on Jun. 30, 2006.
(60) Provisional application No. 60/851,676, filed on Oct. 13, 2006.

(51) Int. Cl. A61K 31/454; A61K 31/415; A61K 31/381; A61K 31/137

(52) U.S. Cl. 514/326; 514/396; 514/443; 514/651

(57) ABSTRACT

The present invention relates to methods of treating obesity, anorexia nervosa, or bulimia nervosa comprising administering a compound of the invention. The present invention further relates to the treatment of metabolic syndrome comprising administering a compound of the invention.

### RELATED APPLICATIONS

This application claims the benefit of U.S. Provisional Application No. 60/775,493, filed Feb. 21, 2006, entitled “TREATMENT OF OBESITY AND EATING DISORDERS WITH NORFLUOXETINE”, U.S. Provisional Application No. 60/790,064, filed Apr. 6, 2006, entitled “CB1 ANTAGONISTS AND INVERSE AGONISTS”, U.S. Provisional Application No. 60/817,835, filed Jun. 30, 2006, entitled “CB1 ANTAGONISTS AND INVERSE AGONISTS”, and U.S. Provisional Application No. 60/851,676, filed Oct. 13, 2006, entitled “TREATMENT OF OBESITY AND EATING DISORDERS WITH NORFLUOXETINE”. The teachings of the above-mentioned applications are incorporated by reference herein in their entirety.

### BACKGROUND

### Obesity

According to the National Health and Nutrition Examination Survey (NHANES III, 1988 to 1994), between one third and one half of men and women in the United States are overweight. In the United States, sixty percent of men and fifty-one percent of women, of the age of 20 or older, are either overweight or obese. In addition, a large percentage of children in the United States are overweight or obese.

Obesity is a condition of complex origin. Increasing evidence suggests that obesity is not a simple problem of self-control but is a complex disorder involving appetite regulation and energy metabolism. In addition, obesity is associated with a variety of conditions associated with increased morbidity and mortality in a population. Although the etiology of obesity is not definitively established, genetic, metabolic, biochemical, cultural and psychosocial factors are believed to contribute. In general, obesity has been described as a condition in which excess body fat puts an individual at a health risk.

There is strong evidence that obesity is associated with increased morbidity and mortality. Disease risk, such as cardiovascular disease risk and type 2 diabetes disease risk, increases independently with increased body mass index (BMI). Indeed, this risk has been quantified as a five percent increase in the risk of cardiac disease for females, and a seven percent increase in the risk of cardiac disease for males, for each point of a BMI greater than 24.9 (Kenchaiah et al., N. Engl. J. Med. 347:305, 2002; Massie, N. Engl. J. Med 347:358, 2002). In addition, there is substantial evidence that weight loss in obese persons reduces important disease risk factors. Even a small weight loss, such as 10% of the initial body weight in both overweight and obese adults has been associated with a decrease in risk factors such as hypertension, hyperlipidemia, and hyperglycemia.

Although diet and exercise provide a simple process to decrease weight gain, overweight and obese individuals often cannot sufficiently control these factors to effectively lose weight. Pharmacotherapy is available; several weight loss drugs have been approved by the Food and Drug Administration that can be used as part of a comprehensive weight loss program. However, many of these drugs have serious adverse side effects. When less invasive methods have failed, and the patient is at high risk for obesity related morbidity or mortality, weight loss surgery is an option in carefully selected patients with clinically severe obesity. However, these treatments are high-risk, and suitable for use in only a limited number of patients.

It is not only obese subjects who wish to lose weight. People with weight within the recommended range, for example, in the upper part of the recommended range, may wish to reduce their weight, to bring it closer to the ideal weight. Thus, a need remains for agents that can be used to effect weight loss in overweight and obese subjects.

### Eating Disorders

Bulimia Nervosa (“ox-like hunger of nervous origin”) was identified as a mental disorder in the early 1970's, but was considered to be an “ominous” variation of the then more recognized eating disorder, anorexia nervosa. Subsequent developments in the study of eating disorders have indicated that, although many anorexia nervosa patients are or may become bulimic, Bulimia Nervosa is a separate disorder with a distinct set of clinically-defined symptoms and behaviors. The disorder anorexia nervosa can be generally characterized by an individual's refusal to maintain a minimally normal body weight usually effectuated through severe restriction of caloric intake. In contrast, Bulimia Nervosa and bulimia-related eating disorders are generally characterized by repeated episodes of binge eating, followed by inappropriate and unhealthy compensatory behaviors such as self-induced vomiting; misuse of laxatives, diuretics, or other medications; fasting or excessive exercise.

Bulimia Nervosa is of unknown etiology, but it affects a relatively large portion of the population. The Diagnostic and Statistical Manual of Eating Disorders, 4^{th} ed., (DSM-IV), reports the prevalence of Bulimia Nervosa to be 1% to 3% within the adolescent and young adult female population, and one-tenth of that in the male population. No reliable statistics are available regarding the prevalence of bulimia-type eating disorders in these populations, but it is believed that the rate is similar, or greater, than that of Bulimia Nervosa. Bulimia Nervosa has been reported to occur with roughly similar frequencies in most industrialized countries, including the United States, Canada, Europe, Australia, Japan, New Zealand and South Africa. Thus, within the female population of industrialized nations, Bulimia Nervosa is at least as common as other major psychiatric disorders such as schizophrenia, which occurs at a rate of 1.5%, and Major Depressive Disorder, which occurs at a rate of 1.3%.

The essential features of Bulimia Nervosa are a disturbance in perception and a high level of preoccupation with body shape and weight, coupled with binge eating and inappropriate compensatory methods to prevent weight gain. Other characteristic behaviors, as well as the physical and psychological symptoms which give rise to a diagnosis of Bulimia Nervosa, are well-known in the art and are detailed in the DSM-IV at pages 545 to 550, the contents of which are incorporated herein by reference.

The diagnostic criteria for Bulimia Nervosa are highly defined; for a diagnosis of Bulimia Nervosa, individuals must exhibit particular behaviors and psychological symptoms with specified frequency. Frequently individuals engaging in disordered eating practices do not meet these DSM-IV criteria, but exhibit behaviors and thought patterns common to individuals diagnosed with Bulimia Nervosa, including binge eating, followed by compensatory behaviors and an undue preoccupation with body shape. These individuals are defined by the DSM-IV as having a Bulimia-Type Eating Disorder Not Otherwise Specified (Eating Disorder N.O.S.). The specific clinical criteria defining Bulimia-Type Eating Disorders N.O.S. are well-known in the art and are detailed in the DSM-IV at page 550, the contents of which are incorporated herein by reference.

The average age for the onset of Bulimia Nervosa or Bulimia-Type Eating Disorder N.O.S. is late adolescence or early childhood. The overwhelming majority of those who are afflicted, approximately 98%, are young women. In a high percentage of cases, the disturbed eating behavior persists for several years. Recovery rates for Bulimia Nervosa have been reported at 38% to 46%. The long-term outcome of Bulimia Nervosa is not known, but anecdotal evidence suggests that relapse is common.

Early epidemiological and family studies of eating disordered individuals demonstrated an apparent linkage betveen such disorders and mood disturbances. This initial observation has been reinforced further by clinical and physiological data. For example, studies of individuals diagnosed with Bulimia Nervosa have indicated a high frequency of comorbid diagnoses of axis I psychiatric disorders, including Major Depressive Disorder. Further, research into the pathophysiological bases of eating disorders has implicated a disturbance in the serotonigenic system of eating disordered individuals, a neurotransmitter system also believed to play a role in mood disorders. Because of the several associations of Bulimia Nervosa and Bulirnia-Type Eating Disorder N.O.S. with mood and anxiety disorders, most of the treatment modalities devised for Bulimia Nervosa and Bulimia-Type Eating Disorder N.O.S. have been developed from, or have been related to, treatment approaches developed for these disorders. In fact, a brief survey of the scientific literature reveals that, although they are not clinically defined as mood or anxiety disorders, Bulimia Nervosa and Bulimia-Type Eating Disorders N.O.S. are frequently treated with antidepressant medications, such as fluoxetine, imipramine and trazodone. There remains a need to treat bulimia nervosa at a dose that does not induce side effects.

Anorexia, defined as the lack or the loss of appetite for food (Dorland's Illustrated Medical Dictionary, 24 edition, W. B. Saunders Company, Philadelphia, 1965) has multiple etiologies. It is commonly associated with cachexia, a state of constitutional disorder, general ill health and malnutrition. Common examples of conditions associated with anorexia and cachexia are anorexia nervosa, certain infectious diseases, and malignancy.

Anorexia nervosa is a serious psychiatric disorder affecting predominantly women (94-96%) in the 13-30 age range. Between 1% (Crisp et al., 128 Br. J. Psychiatry 549, 1976) and 3% (Ballot et al., 59 S. Afr. Med. J. 992, 1981) of young women may be affected. The morbidity and mortality from this condition are considerable. Two years from diagnosis, 4-6% have died and only 50% have achieved a normal weight. There are multiple endocrine and metabolic abnormalities present, most of which are believed to be secondary to the malnutrition. A serious complication of the condition is osteoporosis, which can involve both the spine and peripheral bones. At present there is no specific treatment for anorexia nervosa, although multiple approaches have been tried (Piazza, Piazza & Rollins Compr. Psychiatry 21:177-189 1980). Improved treatments for anorexia are needed.

### Metabolic Syndrome

Metabolic syndrome (also known as “syndrome X,” “dysmetabolic syndrome,” “obesity syndrome,” and “Reaven's syndrome”) has emerged as a growing problem. For example, metabolic syndrome has become increasingly common in the United States. It is estimated that about 47 million adults in the United States have the syndrome.

Metabolic syndrome is generally a constellation of metabolic disorders that all result from, or are associated with, a primary disorder of insulin resistance. Accordingly, the syndrome is sometimes referred to as “insulin resistance syndrome.” Insulin resistance is characterized by disorders in which the body cannot use insulin efficiently and the body's tissues do not respond normally to insulin. As a result, insulin levels become elevated in the body's attempt to overcome the resistance to insulin. The elevated insulin levels lead, directly or indirectly, to the other metabolic abnormalities.

Some people are genetically predisposed to insulin resistance, while other people acquire factors that lead to insulin resistance. Acquired factors, such as excess body fat and physical inactivity, can elicit insulin resistance, and more broadly, clinical metabolic syndrome. Because of this relationship between insulin resistance and metabolic syndrome, it is believed that the underlying causes of this syndrome are obesity, physical inactivity and genetic factors. In fact, most people with insulin resistance and metabolic syndrome have central obesity (excessive fat tissue in and around the abdomen). The biologic mechanisms at the molecular level between insulin resistance and metabolic risk factors are not yet fully understood and appear to be complex.

Metabolic syndrome is typically characterized by a group of metabolic risk factors that include 1) central obesity; 2) atherogenic dyslipidemia (blood fat disorders comprising mainly high triglycerides (“TG”) and low HDL-cholesterol (interchangeably referred to herein as “HDL”) that foster plaque buildups in artery walls); 3) raised blood pressure; 4) insulin resistance or glucose intolerance (the body can't properly use insulin or blood sugar); 5) prothrombotic state (e.g., high fibrinogen or plasminogen activator inhibitor in the blood); and 6) a proinflammatory state (e.g., elevated-high-sensitivity C-reactive protein in the blood). The National Cholesterol Education Program (NCEP) Adult Treatment Panel (ATP) III guidelines define metabolic syndrome by the following five clinical parameters: a) a waist circumference greater than 102 cm for men, and greater than 88 cm for women; b) a triglyceride level greater than 150 mg/dl; c) an HDL-cholesterol less than 40 mg/dl for men, and less than 50 mg/dl for women; d) a blood pressure greater than or equal to 130/85 mmHg; and e) a fasting glucose greater than 110 mg/dl.

According to the American Heart Association, however, there are no well-accepted criteria for diagnosing metabolic syndrome. Some guidelines suggest that metabolic syndrome involves four general factors: obesity; diabetes; hypertension; and high lipids. According to the NCEP ATP III guidelines above, the presence of at least three of these five factors meets the medical diagnosis of metabolic syndrome.

Although there is no complete agreement on the individual risk or prevalence of each factor, it is known that the syndrome, as generally agreed upon by those skilled in the field, poses a significant health risk to individuals. A person having one factor associated with the syndrome has an increased risk for having one or more of the others. The more factors that are present, the greater the risks to the person's health. When the factors are present as a group, i.e., metabolic syndrome, the risk for cardiovascular disease and premature death is very high.

For example, a person with the metabolic syndrome is at an increased risk of coronary heart disease, other diseases related to plaque buildups in artery walls (e.g., stroke and peripheral vascular disease), prostate cancer, and type 2 diabetes. It is also known that when diabetes occurs, the high risk of cardiovascular complications increases.

Generally, patients suffering from the syndrome are prescribed a change in lifestyle, e.g., an increase in exercise and a change to a healthy diet. The goal of exercise and diet programs is to reduce body weight to within 20% of the “ideal” body weight calculated for age and height.

In some cases, diet and exercise regimens are supplemented with treatments for lipid abnormalities, clotting disorders, and hypertension. For example, patients with the syndrome typically have several disorders of coagulation that make it easier to form blood clots within blood vessels. These blood clots are often a precipitating factor in developing heart attacks. Patients with the syndrome are often placed on daily aspirin therapy to specifically help prevent such clotting events. Furthermore, high blood pressure is present in more than half the people with the syndrome, and in the setting of insulin resistance, high blood pressure is especially important as a risk factor. Some studies have suggested that successfully treating hypertension in patients with diabetes can reduce the risk of death and heart disease by a substantial amount. Additionally, patients have been treated to specifically reduce LDL-cholesterol (interchangeably referred to herein as “LDL”) levels, reduce triglyceride levels, and raise HDL levels. Given the increasing prevalence of this syndrome, there remains a need for additional and effective treatments of the syndrome.

### SUMMARY OF INVENTION

The present invention relates to a method of treating obesity in a mammal. The invention further relates to a method of minimizing metabolic risk factors associated with obesity, such as hypertension, diabetes and dyslipidemia. In one embodiment, the methods comprise administering to a mammal in need of such treatment an effective anti-obesity dose of a compound of any one of formulae 1-6 or a salt thereof, or a solvate of the compound or its salt. In another embodiment, the methods comprise administering to a mammal in need of such treatment an effective anti-obesity dose of norfluoxetine or a salt thereof or a solvate of norfluoxetine or its salt. In certain such embodiments, the norfluoxetine is (R)-norfluoxetine.

The present invention also relates to a method of treating anorexia nervosa in a mammal. In one embodiment, the methods comprise administering to a mammal in need of treatment of anorexia nervosa an effective amount of a compound of any one of formulae 1-6 or a salt thereof, or a solvate of the compound or its salt. In another embodiment, the methods comprise administering to a mammal in need of such treatment an effective amount of norfluoxetine or a salt thereof, or a solvate of norfluoxetine or its salt. In certain such embodiments, the norfluoxetine is (R)-norfluoxetine.

The present invention also relates to a method of treating bulimia nervosa or a bulimia-type eating disorder not otherwise specified in a mammal. In one embodiment, the methods comprise administering to a mammal in need of treatment of bulimia nervosa or a bulimia-type eating disorder not otherwise specified an effective amount of a compound of any one of formulae 1-6 or a salt thereof, or a solvate of the compound or its salt. In another embodiment, the methods comprise administering to a mammal in need of such treatment an effective amount of norfluoxetine or a salt thereof, or a solvate of norfluoxetine or its salt. In certain such embodiments, the norfluoxetine is (R)-norfluoxetine.

In another aspect, the present invention provides a method of treating obesity, anorexia nervosa, bulimia nervosa, a bulimia-type eating disorder not otherwise specified, metabolic syndrome or a disorder associated with metabolic syndrome (e.g., obesity, diabetes, hypertension, and hyperlipidemia) in a mammal comprising administering to a mammal suffering from obesity, anorexia nervosa, bulimia nervosa, a bulimia-type eating disorder not otherwise specified, metabolic syndrome or a disorder associated with metabolic syndrome (e.g., obesity, diabetes, hypertension, and hyperlipidemia) a CB 1 antagonist or inverse agonist conjointly with an allosteric potentiator of MC4, an agonist of MC4, an inhibitor of dopamine reuptake, an inhibitor of norepinephrine reuptake, an inhibitor of both dopamine and norepinephrine reuptake, an MAO-B inhibitor, a dopamine D1 agonist, a dopamine D2 agonist, a dopamine D3 agonist, a dopamine D4 agonist, or a dopamine D5 agonist. In certain embodiments, the CB1 antagonist or inverse agonist is administered conjointly with a D2 agonist.

In another aspect, the present invention provides a method of treating obesity, anorexia nervosa, bulimia nervosa, a bulimia-type eating disorder not otherwise specified, metabolic syndrome or a disorder associated with metabolic syndrome (e.g., obesity, diabetes, hypertension, and hyperlipidemia) in a mammal comprising administering to a mammal suffering from obesity, anorexia nervosa, bulimia nervosa, a bulimia-type eating disorder not otherwise specified, metabolic syndrome or a disorder associated with metabolic syndrome (e.g., obesity, diabetes, hypertension, and hyperlipidemia) a CB1 antagonist or inverse agonist conjointly with bupropion, methylphenidate, sibutramine, sertraline, venlafaxine, atomoxetine, amineptine, benztropine, reboxetine, rasagiline, selegiline, deprenyl, lazabemide, quinpirole, talipexole, sumanirole, bromocriptine, ropinirole, pramipexole, levodopa (optionally in combination with carbidopa), amantadine, pergolide, fenoldopam, cabergoline, rotigotine, lysuride, 7-OH DPAT, SKF-38393, apomorphine, or a pharmaceutically acceptable salt, metabolite or stereoisomer thereof. In certain embodiments, the CB1 antagonist or inverse agonist is conjointly administered with bupropion or a pharmaceutically acceptable salt, metabolite, or stercoisomer thereof for the treatment of anorexia nervosa, bulimia nervosa, or a bulimia-type eating disorder not otherwise specified.

In preferred embodiments of the methods of the invention, the mammal is a human.

In another aspect, the present invention provides a method of treating obesity in a patient in need of anti-psychotic treatment, comprising administering to said patient a CB1 antagonist or inverse agonist. In another aspect, the present invention provides a method of treating obesity in a patient being treated with one or more anti-psychotic agents comprising administering to said patient a CB1 antagonist or inverse agonist.

In certain embodiments, the CB1 antagonist or inverse agonist is a compound of any one of formulae 1-6 or a salt thereof, or a solvate of the compound or its salt. In certain embodiments, the CB1 antagonist or inverse agonist is norfluoxetine enriched for the (R) enantiomer.

The present invention also relates to a method of treating prostate cancer in a mammal. In one embodiment, the methods comprise administering to a mammal in need of treatment of prostate cancer an effective amount of norfluoxetine. In certain such embodiments, the norfluoxetine is (R)-norfluoxetine.

### BRIEF DESCRIPTION OF THE DRAWINGS

FIG. 1A shows the Ca²⁺ᵢ emissions of cells loaded with Indo-1.

FIG. 1B shows CB1 cells that responded to 2-AG with an increase in Ca²⁺ᵢ.

FIG. 2 shows the potential CB1 attenuator/antagonist activity of compound 7 and the selectivity for the CB1 receptor over the CB2 receptor.

FIG. 3 shows the lack of effect compound 7 has on the dissociation rate of [³H] CP 55940.

FIG. 4 shows the pA2 estimation of compound 7 using CP 55940 as an agonist with the CB1⁹⁰ cell line.

FIG. 5 shows the [³⁵S]GTPγS binding assay of compound 7.

FIG. 6 shows the binding properties of compound 7 in human and mouse CB1 receptors.

FIG. 7 shows inhibition of agonist and antagonist binding to mouse CB1 by compound 7.

FIG. 8 shows the results of oral administration of Treatment X, Treatment Y, Sibutramine and Rimonabant on the body weight of diet-induced obese mice.

FIG. 9 shows the effect of Treatment A and rimonabant on the consumption of wet mash in lean male C57BLJ6J mice.

FIG. 10 shows the effect of Treatment A and rimonabant on 24 hour body weight change in lean male C57BL/6J mice.

FIG. 11 shows the effect of Treatment B and rimonabant on the consumption of wet mash in lean male C57BL/6J mice.

FIG. 12 shows the effect of Treatment B and rimonabant on 24 hour body weight change in lean male C57BL/6J mice.

FIG. 13 shows the effect of Treatment C and rimonabant on the consumption of wet mash in lean male C57BL/6J mice.

FIG. 14 shows interval data for the effect of Treatment C and rimonabant on the consumption of wet mash in lean male C57BL/6J mice.

FIG. 15 shows the effect of Treatment C and rimonabant on daily food intake of lean male C57BL/6J mice.

FIG. 16 shows the effect of Treatment C and rimonabant on the body weight of lean male C57BL/6J mice.

FIG. 17 shows the effect of Treatment C and rimonabant on 24 hour body weight change in lean male C57BL/6J mice.

FIG. 18 shows the effect of Treatment D and rimonabant on the consumption of wet mash in lean male C57BL/6J mice.

FIG. 19 shows interval data for the effect of Treatment D and rimonabant on the consumption of wet mash in lean male C57BL/6J mice.

FIG. 20 shows the effect of Treatment D and rimonabant on daily food intake of lean male C57BL/6J mice.

FIG. 21 shows the effect of Treatment D and rimonabant on 24 hour body weight change in lean male C57BL/6J mice.

### DETAILED DESCRIPTION OF THE INVENTION

The present invention relates to a method of treating obesity in a mammal. The invention further relates to a method of minimizing metabolic risk factors associated with obesity, such as hypertension, diabetes and dyslipidemia. In one embodiment, the methods comprise administering to a mammal in need of such treatment an effective anti-obesity dose of a compound of any one of formulae 1-6. In another embodiment, the methods comprise administering to a mammal in need of such treatment an effective anti-obesity dose of norfluoxetine. In certain such embodiments, the norfluxoetine is (R)-norfluoxetine.

The present invention also relates to a method of treating anorexia nervosa in a mammal. In one embodiment, the methods comprise administering to a mammal in need of treatment of anorexia nervosa an effective amount of a compound of any one of formulae 1-6. In another embodiment, the methods comprise administering to a mammal in need of such treatment an effective amount of norfluoxetine. In certain such embodiments, the norfluoxetine is (R)-norfluoxetine.

The present invention also relates to a method of treating bulimia nervosa or a bulimia-type eating disorder not otherwise specified in a mammal. In one embodiment, the methods comprise administering to a mammal in need of treatment of bulimia nervosa or a bulimia-type eating disorder not otherwise specified an effective amount of a compound of any one of formulae 1-6. In another embodiment, the methods comprise administering to a mammal in need of such treatment an effective amount of norfluoxetine. In certain such embodiments, the norfluoxetine is (R)-norfluoxetine.

In certain embodiments of methods of the invention wherein an effective amount of norfluoxetine is administered for the treatment of obesity, anorexia nervosa, bulimia nervosa, or a bulimia-type eating disorder not otherwise specified, it is contemplated that a therapeutically effective amount (dose) of the compound (e.g., norfluoxetine) to be administered to a subject (e.g., a mammal, preferably a human) will be in the range of 1 mg/day to 100 mg/day, 1 mg/day to 60 mg/day, 1 mg/day to 40 mg/day, or even 1 mg/day to 10 mg/day. In general, the therapeutically effective dose for the treatment of obesity, anorexia nervosa, bulimia nervosa, or a bulimia-type eating disorder not otherwise specified is less than the therapeutically effective dose for the treatment of major depressive disorder or obsessive compulsive disorder.

In another aspect, the present invention provides a method of treating obesity, anorexia nervosa, bulimia nervosa, or a bulimia-type eating disorder not otherwise specified in a mammal comprising administering to a mammal suffering from obesity, anorexia nervosa, bulimia nervosa, or a bulimia-type eating disorder not otherwise specified a CB1 antagonist or inverse agonist conjointly with an allosteric potentiator of MC4, an agonist of MC4, an inhibitor of dopamine reuptake, an inhibitor of norepinephrine reuptake, an inhibitor of both dopamine and norepinephrine reuptake, an MAO-B inhibitor, a dopamine D1 agonist, a dopamine D2 agonist, a dopamine D3 agonist, a dopamine D4 agonist, or a dopamine D5 agonist. In certain embodiments, the CB1 antagonist or inverse agonist is administered conjointly with a D2 agonist.

In certain embodiments, an agonist or antagonist as described above (e.g., dopamine agonists) may be either a full or partial agonist or antagonist.

In certain embodiments, the CB1 antagonist or inverse agonist is a compound of any one of formulae 1-6. In certain embodiments, the CB1 antagonist or inverse agonist is rimonabant, LH-21, fluoxetine, norfluoxetine, or a pharmaceutically acceptable salt thereof. In certain embodiments, the CB1 antagonist or inverse agonist is norfluoxetine enriched for the (R) enantiomer.

In another aspect, the present invention provides a method of treating obesity, anorexia nervosa, bulimia nervosa, or a bulimia-type eating disorder not otherwise specified in a mammal comprising administering to a mammal suffering from obesity, anorexia nervosa, bulimia nervosa, or a bulimia-type eating disorder not otherwise specified a CB1 antagonist or inverse agonist conjointly with methylphenidate, sibutramine, sertraline, venlafaxine, atomoxetine, amineptine, benztropine, reboxetine, rasagiline, selegiline, deprenyl, lazabemide, quinpirole, talipexole, sumanirole, bromocriptine, ropinirole, pramipexole, levodopa (optionally in combination with carbidopa), amantadine, pergolide, fenoldopam, cabergoline, rotigotine, lysuride, 7-OH DPAT, SKF-38393, or apomorphine.

In another aspect, the present invention provides a method of treating anorexia nervosa, bulimia nervosa, or a bulimia-type eating disorder not otherwise specified in a mammal comprising administering to a mammal suffering from anorexia nervosa, bulimia nervosa, or a bulimia-type eating disorder not otherwise specified a CB1 antagonist or inverse agonist conjointly with bupropion or a pharmaceutically acceptable salt, metabolite, or stereoisomer thereof.

As used herein, unless otherwise indicated, the generic name of a drug is used to signify a chemical compound and its pharmaceutically acceptable salts and enantiomeric forms. For example, the term “bupropion” will be used to include any acid addition salt, the free base, the racemic mixture, and the purified (R) and (S) enantiomers.

In certain embodiments, the CB1 antagonist or inverse agonist is a compound of any one of formulae 1-6. In certain embodiments, the CB1 antagonist or inverse agonist is rimonabant, LH-21, fluoxetine, norfluoxetine, or a pharmaceutically acceptable salt thereof. In certain embodiments, the CB1 antagonist or inverse agonist is norfluoxetine enriched for the (R) enantiomer.

In certain embodiments, the present invention provides a method of treating obesity, anorexia nervosa, bulimia nervosa. or a bulimia-type eating disorder not otherwise specified in a mammal comprising administering to a mammal suffering from obesity, anorexia nervosa, bulimia nervosa, or a bulimia-type eating disorder not otherwise specified norfluoxetine enriched for the (R) enantiomer conjointly with bupropion. In certain embodiments of methods of the invention wherein norfluoxetine enriched for the (R) enantiomer is administered conjointly with bupropion for the treatment of obesity, anorexia nervosa, bulimia nervosa, or a bulimia-type eating disorder not otherwise specified, the method further comprises administering moxonidine or a pharmaceutically acceptable salt thereof.

In certain embodiments of methods of the invention wherein norfluoxetine enriched for the (R) enantiomer is administered conjointly with bupropion, the norfluoxetine enriched for the (R) enantiomer and bupropion are administered in a molar ratio in the range of 1:1 to 20:1, 2:1 to 20:1, 4:1 to 20:1, or even 6:1 to 20:1.

In certain embodiments of methods of the invention wherein norfluoxetine enriched for the (R) enantiomer is administered conjointly with bupropion, (R)-norfluoxetine (D)-tartrate and bupropion hydrochloride are administered in a weight ratio in the range of 1:1 to 20:1, 4:1 to 20:1, 6:1 to 20:1, or even 10:1 to 20:1.

In certain embodiments of methods of the invention wherein norfluoxetine enriched for the (R) enantiomer is administered conjointly with bupropion, (R)-norfluoxetine hydrochloride and bupropion hydrochloride are administered in a weight ratio in the range of 1:1 to 20:1, 2:1 to 20:1, 4:1 to 20:1, or even 6:1 to 20:1.

In certain embodiments of methods of the invention wherein norfluoxetine enriched for the (R) enantiomer is administered conjointly with bupropion for the treatment of obesity, the mammal is not also undergoing smoking cessation.

In another aspect, the present invention provides a method of treating obesity, anorexia nervosa, bulimia nervosa, or a bulimia-type eating disorder not otherwise specified in a mammal comprising administering to a mammal suffering from obesity, anorexia nervosa, bulimia nervosa, or a bulimia-type eating disorder not otherwise specified norfluoxetine enriched for the (R) enantiomer conjointly with moxonidine or a pharmaceutically acceptable salt thereof.

In preferred embodiments of the methods of the invention, the mammal is a human.

In another aspect, the present invention provides a method of treating or preventing metabolic syndrome or a disorder associated with metabolic syndrome (e.g., obesity, diabetes, hypertension, and hyperlipidemia) in a mammal comprising administering to a mammal suffering from metabolic syndrome or a disorder associated with metabolic syndrome (e.g., obesity, diabetes, hypertension, and hyperlipidemia) a CB1 antagonist or inverse agonist conjointly with an allosteric potentiator of MC4, an agonist of MC4, an inhibitor of dopamine reuptake, an inhibitor of norepinephrine reuptake, an inhibitor of both dopamine and norepinephrine reuptake, an MAO-B inhibitor, a dopamine D1 agonist, a dopamine D2 agonist, a dopamine D3 agonist, a dopamine D4 agonist, or a dopamine D5 agonist. In certain embodiments, the CB1 antagonist or inverse agonist is administered conjointly with a D2 agonist.

In certain embodiments, agonist or antagonist as described above (e.g., dopamine agonists) may be either full or partial agonists or antagonists.

In certain embodiments, the CB1 antagonist or inverse agonist is a compound of any one of formulae 1-6. In certain embodiments, the CB1 antagonist or inverse agonist is rimonabant, LH-21, fluoxetine, norfluoxetine, or a pharmaceutically acceptable salt thereof. In certain embodiments, the CB1 antagonist or inverse agonist is norfluoxetine enriched for the (R) enantiomer.

In another aspect, the present invention provides a method of treating or preventing metabolic syndrome or a disorder associated with metabolic syndrome (e.g., obesity, diabetes, hypertension, and hyperlipidemia) in a mammal comprising administering to a mammal suffering from metabolic syndrome or a disorder associated with metabolic syndrome (e.g., obesity, diabetes, hypertension, and hyperlipidemia) a CB1 antagonist or inverse agonist conjointly with bupropion, methylphenidate, sibutramine, sertraline, venlafaxine, atomoxetine, amineptine, benztropine, reboxetine, rasagi line, selegiline, deprenyl, lazabemide, quinpirole, talipexole, sumanirole, bromocriptine, ropinirole, pramipexole, levodopa (optionally in combination with carbidopa), amantadine, pergolide, fenoldopam, cabergoline, rotigotine, lysuride, 7-OH DPAT, SKF-38393, or apomorphine. In certain embodiments, the CB1 antagonist or inverse agonist is conjointly administered with bupropion or a pharmaceutically acceptable salt, metabolite, or stereoisomer thereof. In certain embodiments wherein the CB1 antagonist or inverse agonist is conjointly administered with bupropion or a pharmaceutically acceptable salt, metabolite, or stereoisomer thereof for the treatment or prevention of a disorder associated with metabolic syndrome, the disorder is not obesity.

In certain embodiments, the CB1 antagonist or inverse agonist is a compound of any one of formulae 1-6. In certain embodiments, the CB1 antagonist or inverse agonist is rimonabant, LH-21, fluoxetine, norfluoxetine, or a pharmaceutically acceptable salt thereof. In certain embodiments, the CB1 antagonist or inverse agonist is norfluoxetine enriched for the (R) enantiomer.

In certain embodiments, the present invention provides a method of treating or preventing metabolic syndrome or a disorder associated with metabolic syndrome (e.g., obesity, diabetes, hypertension, and hyperlipidemia) in a mammal comprising administering to a mammal suffering from metabolic syndrome or a disorder associated with metabolic syndrome (e.g., obesity, diabetes, hypertension, and hyperlipidemia) norfluoxetine enriched for the (R) enantiomer conjointly with bupropion. In certain embodiments of methods of the invention wherein norfluoxetine enriched for the (R) enantiomer is administered conjointly with bupropion for the treatment or prevention of metabolic syndrome or a disorder associated with metabolic syndrome (e.g., obesity, diabetes, hypertension, and hyperlipidemia), the method further comprises administering moxonidine or a pharmaceutically acceptable salt thereof.

In preferred embodiments of the methods of the invention, the mammal is a human.

In certain embodiments, the therapeutic dose of the CB1 antagonist or inverse agonist when administered conjointly with an allosteric potentiator of MC4, an agonist of MC4, an inhibitor of dopamine reuptake, an inhibitor of norepinephrine reuptake, an inhibitor of both dopamine and norepinephrine reuptake, an MAO-B inhibitor, a dopamine D1 agonist, a dopamine D2 agonist, a dopamine D3 agonist, a dopamine D4 agonist, or a dopamine DS agonist is less than that required for a therapeutic dose when administered alone. In certain embodiments, the therapeutic dose of the allosteric potentiator of MC4, the agonist of MC4, the inhibitor of dopamine reuptake, the inhibitor of norepinephrine reuptake, the inhibitor of both dopamine and norepinephrine reuptake, the MAO-B inhibitor, the dopamine D1 agonist, the dopamine D2 agonist, the dopamine D3 agonist, the dopamine D4 agonist, or the dopamine D5 agonist when administered conjointly with a CB1 antagonist or inverse agonist is less than that required for a therapeutic dose when administered alone.

In another aspect, the present invention provides a method of treating or preventing metabolic syndrome or a disorder associated with metabolic syndrome (e.g., obesity, diabetes, hypertension, and hyperlipidemia) in a mammal comprising administering to a mammal suffering from metabolic syndrome or a disorder associated with metabolic syndrome (e.g., obesity, diabetes, hypertension, and hyperlipidemia) norfluoxetine enriched for the (R) enantiomer conjointly with moxonidine or a pharmaceutically acceptable salt thereof.

In preferred embodiments of the methods of the invention, the mammal is a human.

The present invention also relates to a method of treating prostate cancer in a mammal. In one embodiment, the methods comprise administering to a mammal in need of treatment of prostate cancer an effective amount of norfluoxetine. In certain such embodiments, the norfluoxetine is (R)-norfluoxetine. In certain embodiments of methods of the invention wherein an effective amount of norfluoxetine is administered for the treatment of prostate cancer, it is contemplated that a therapeutically effective amount (dose) of the compound (e.g., norfluoxetine) to be administered to a subject (e.g., a mammal, preferably a human) will be in the range of 1 mg/day to 100 mg/day, 1 mg/day to 60 mg/day, 1 mg/day to 40 mg/day, or even 1 mg/day to 10 mg/day. In general, the therapeutically effective dose for the treatment of prostate cancer is less than the therapeutically effective dose for the treatment of major depressive disorder or obsessive compulsive disorder.

In another aspect, the present invention provides a method of treating obesity in a patient in need of anti-psychotic treatment, comprising administering to said patient a CB1 antagonist or inverse agonist. In another aspect, the present invention provides a method of treating obesity in a patient being treated with one or more anti-psychotic agents, comprising administering to said patient a CB1 antagonist or inverse agonist.

In certain embodiments, the CB1 antagonist or inverse agonist is a compound of any one of formulae 1-6. In certain embodiments, the CB1 antagonist or inverse agonist is rimonabant, LH-21, fluoxetine, norfluoxetine, or a pharmaceutically acceptable salt thereof. In certain embodiments, the CB1 antagonist or inverse agonist is norfluoxetine enriched for the (R) enantiomer.

In certain embodiments of methods of the invention wherein a CB1 antagonist or inverse agonist is administered to a patient being treated with one or more anti-psychotic agents, the anti-psychotic agents are selected from any suitable anti-psychotic agent. Suitable anti-psychotic agents include, but are not limited to, clozapine, olanzapine, quetiapine, risperidone, ziprasidone, aripiprazole, trifluoperazine, flupenthixol, loxapine, perphenazine, chlorpromazine, haloperidol, fluphenazine decanoate, thioridazine, or a pharmaceutically acceptable salt thereof.

In certain embodiments, the present invention relates to methods of treatment with norfluoxetine. In certain embodiments, the therapeutic preparation may be enriched to provide predominantly one enantiomer of norfluoxetine. An enantiomerically enriched mixture may comprise, for example, at least 60 mol percent of one enantiomer, or more preferably at least 75, 90, 95, or even 99 mol percent. In certain embodiments, norfluoxetine is enriched in the (R) enantiomer. In certain embodiments, (R)-norfluoxetine is substantially free of the (S)-enantiomer, wherein substantially free means that the substance in question makes up less than 10%, or less than 5%, or less than 4%, or less than 3%, or less than 2%, or less than 1% as compared to the amount of the (R)-enantiomer, e.g., in the composition or compound mixture. For example, if a composition or compound mixture contains 98 grams of the (R)-enantiomer and 2 grams of the (S)-enantiomer, it would be said to contain 98 mol percent of the (R)-enantiomer and only 2% of the (S)-enantiomer. In certain embodiments, norfluoxetine is provided as a salt of norfluoxetine or a solvate of norfluoxetine or its salt.

Fluoxetine is a racemate of two enantiomeric forms. Early reports cited that the biological and pharmacological activity of each enantiomer, as relates to interaction with the serotonin uptake carrier, was found to be essentially the same; see, Robertson et al., J. Med. Chem., 31, 1412 (1988) and references cited therein. Norfluoxetine [3-(4-trifluoromethylphenoxy)-3-phenylpropylamine] is a metabolite of fluoxetine and is known to block monoamine uptake, especially serotonin. See U.S. Pat. No. 4,313,896. Since norfluoxetine it is a metabolite of fluoxetine, it is believed that this compound contributes in part to the biological activity seen upon administration of fluoxetine.

Both fluoxetine and notfluoxetine exhibit functional activity versus the CB1 receptor. (S)-Fluoxetine is an inverse agonist of CB1, and (R)-fluoxetine is an antagonist of CB1. The racemate of norfluoxetine is an antagonist of CB1. Without wishing to be restricted by the proposal, this cannabinoid activity may mediate the utility of these compounds for the treatment of obesity and/or eating disorders.

Compounds suitable for use in methods of the invention include compounds represented by general formula 1:

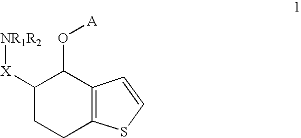

wherein
A represents a substituted or unsubstituted aryl or heteroaryl ring;
R₁ and R₂ are each independently for each occurrence selected from H or substituted or unsubstituted C₁₋₆alkyl, C₁₋₆aralkyl, aryl, heteroaryl, or acyl, or R₁ and R₂ taken together with the N to which they are bound form a substituted or unsubstituted 5- to 7-membered cyclic or heterocyclic ring system; and
X represents a substituted or unsubstituted methylene.

Compounds suitable for use in methods of the invention include compounds represented by the general formula 2:

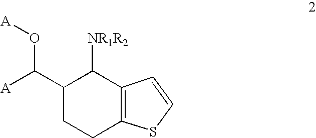

wherein
A independently for each occurrence represents a substituted or unsubstituted aryl or heteroaryl ring; and
R₁ and R₂ are each independently for each occurrence selected from H or substituted or unsubstituted C₁₋₆alkyl, C₁₋₆aralkyl, aryl, heteroaryl, or acyl, or R₁ and R₂ taken together with the N to which they are bound form a substituted or unsubstituted 5- to 7-membered cyclic or heterocyclic ring system.

Compounds suitable for use in methods of the invention include compounds represented by general formula 3:

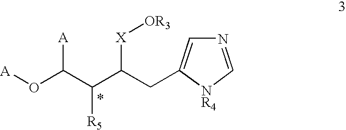

wherein
A independently for each occurrence represents a substituted or unsubstituted aryl or heteroaryl ring;
X represents a substituted or unsubstituted methylene;
R₃ represents H or substituted or unsubstituted C₁₋₆alkyl, C₁₋₆aralkyl, aryl, heteroaryl, or acyl;
R₄ represents H or substituted or unsubstituted C₁₋₆alkyl; and
R₅ represents substituted or unsubstituted C₁₋₆alkyl, acyl, C₁₋₆aralkyl, aryl, heteroaryl, carbocycle, or heterocycle, provided that when R₅ is substituted or unsubstituted heteroaryl or heterocycle, the atom that is attached to the indicated (*) carbon is a carbon atom.

Compounds suitable for use in methods of the invention include compounds represented by general formula 4:

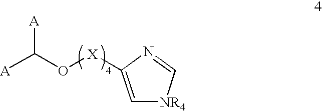

wherein
A independently for each occurrence represents a substituted or unsubstituted aryl or heteroaryl ring;
X independently for each occurrence represents a substituted or unsubstituted methylene; and
R₄ represents H or substituted or unsubstituted C₁₋₆alkyl.

Compounds suitable for use in methods of the invention include compounds represented by general formula 5:

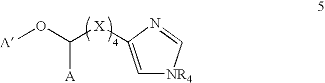

wherein
A represents a substituted or unsubstituted aryl or heteroaryl ring;
A′ represents a substituted aryl or heteroaryl ring;
X independently for each occurrence represents a substituted or unsubstituted methylene; and
R₄ represents H or substituted or unsubstituted C₁₋₆alkyl.

Compounds suitable for use in methods of the invention include compounds represented by general formula 6

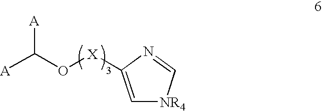

wherein
A independently for each occurrence represents a substituted or unsubstituted aryl or heteroaryl ring;
X independently for each occurrence represents a substituted or unsubstituted methylene; and
R₄ represents H or substituted or unsubstituted C₁₋₆alkyl.

In certain embodiments, a compound of formula 6 has the structure 6a or 7:

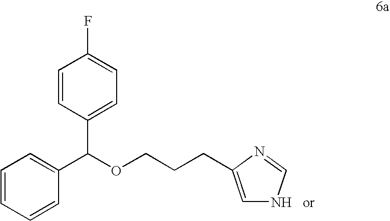

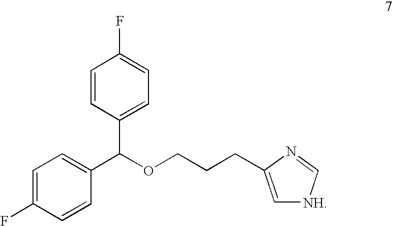

The present invention also relates to certain novel compounds, including purified preparations of those compounds. For instance, the invention provides compounds of formula 1:

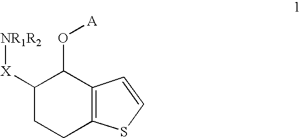

wherein
A represents a substituted or unsubstituted aryl or heteroaryl ring;
R₁ and R₂ are each independently for each occurrence selected from H or substituted or unsubstituted C₁₋₆alkyl, C₁₋₆aralkyl, aryl, heteroaryl, or acyl, or R₁ and R₂ taken together with the N to which they are bound form a substituted or unsubstituted 5- to 7-membered cyclic or heterocyclic ring system; and
X represents a substituted or unsubstituted methylene.

In certain embodiments, R₁ is hydrogen and R₂ is substituted or unsubstituted C₁₋₆alkyl, preferably methyl.

In certain embodiments, A is a substituted aryl ring. In certain embodiments, A is p-trifluoromethylphenyl.

In certain embodiments, a compound of formula 1 has the structure 1a:

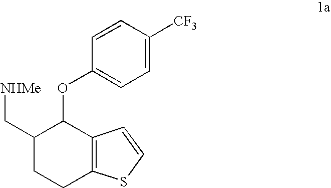

In certain embodiments, the compound can be represented by the general formula 2:

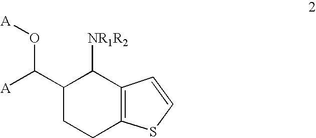

wherein
A independently for each occurrence represents a substituted or unsubstituted aryl or heteroaryl ring; and
R₁ and R₂ are each independently for each occurrence selected from H or substituted or unsubstituted C₁₋₆alkyl, C₁₋₆aralkyl, aryl, heteroaryl, or acyl, or R₁ and R₂ taken together with the N to which they are bound form a substituted or unsubstituted 5- to 7-membered cyclic or heterocyclic ring system.

In certain embodiments, R₁ is hydrogen and R₂ is substituted or unsubstituted C₁₋₆alkyl, preferably methyl.

In certain embodiments, A is substituted or unsubstituted aryl.

In certain embodiments, a compound of formula 2 has the structure 2a or 2b:

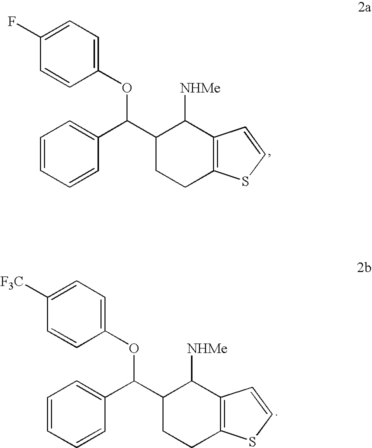

In certain embodiments, the compound can be represented by the general formula 3:

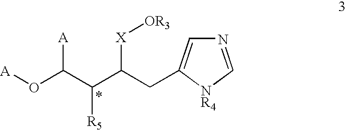

wherein
A independently for each occurrence represents a substituted or unsubstituted aryl or heteroaryl ring;
X represents a substituted or unsubstituted methylene;
R₃ represents H or substituted or unsubstituted C₁₋₆alkyl, C₁₋₆aralkyl, aryl, heteroaryl, or acyl;
R₄ represents H or substituted or unsubstituted C₁₋₆alkyl; and
R₅ represents substituted or unsubstituted C₁₋₆alkyl, acyl, C₁₋₆aralkyl, aryl, heteroaryl, carbocycle, or heterocycle, provided that when R₅ is substituted or unsubstituted heteroaryl or heterocycle, the atom that is attached to the indicated (*) carbon is a carbon atom.

In certain embodiments, A is a substituted or unsubstituted aryl ring.

In certain embodiments, X is an unsubstituted methylene.

In certain embodiments, R₃ represents substituted or unsubstituted C₁₋₆alkyl, preferably methyl.

In certain embodiments, R₄ represents substituted or unsubstituted C₁₋₆alkyl, preferably methyl.

In certain embodiments, R₅ represents substituted or unsubstituted C₁₋₆alkyl, preferably ethyl.

In certain embodiments, a compound of formula 3 has the structure 3a or 3b:

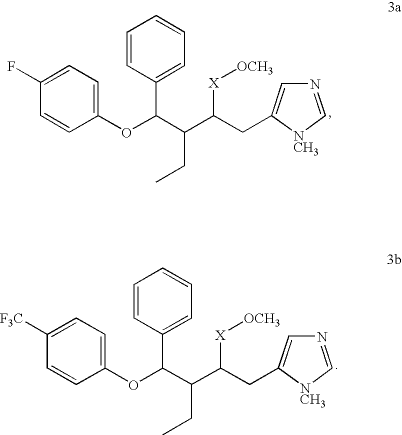

In certain embodiments, the compound can be represented by the general formula 4:

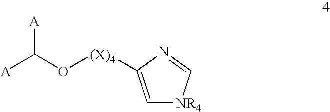

wherein
A independently for each occurrence represents a substituted or unsubstituted aryl or heteroaryl ring;
X independently for each occurrence represents a substituted or unsubstituted methylene; and
R₄ represents H or substituted or unsubstituted C₁₋₆alkyl.

In certain embodiments, R₄ is H.

In certain embodiments, A is a substituted or unsubstituted aryl ring.

In certain embodiments, X is an unsubstituted methylene.

In certain embodiments, a compound of formula 4 has the structure 4a:

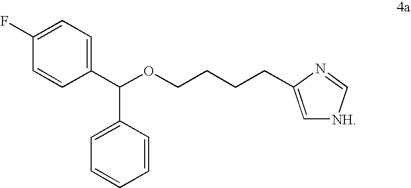

In certain embodiments, the compound can be represented by the general formula 5:

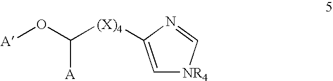

wherein
A represents a substituted or unsubstituted aryl or heteroaryl ring;
A′ represents a substituted aryl or heteroaryl ring;
X independently for each occurrence represents a substituted or unsubstituted methylene; and
R₄ represents H or substituted or unsubstituted C₁₋₆alkyl.

In certain embodiments, R₄ is H.

In certain embodiments, A is a substituted or unsubstituted aryl ring.

In certain embodiments, X is an unsubstituted methylene.

In certain embodiments, a compound of formula 5 has the structure 5a:

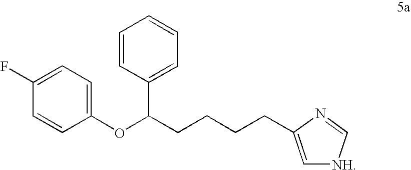

In certain embodiments, compounds of the invention may be racemic. In certain embodiments, compounds of the invention may be enriched in one enantiomer. For example, a compound of the invention may have greater than 30% ee, or 40% ee, or 50% ee, or 60% ee, or 70% ee, or 80% ee, or 90% ee, or even 95% or greater ee. In certain embodiments, compounds of the invention may be enriched in one or more diastereomer. For example, a compound of the invention may have greater than 30% de, or 40% de, or 50% de, or 60% de, or 70% de, or 80% de, or 90% de, or even 95% or greater de.

One aspect of the present invention provides a pharmaceutical composition suitable for use in a human patient, or for veterinary use, comprising an effective amount of a compound of the invention (e.g., a compound of any one of formulae 1-6 or norfluoxetine enriched for the (R) enantiomer), and one or more pharmaceutically acceptable carriers. In certain embodiments, the pharmaceutical compositions may be for use in treating or preventing obesity, anorexia nervosa, bulimia nervosa, or a bulimia-type eating disorder not otherwise specified. In certain embodiments, the pharmaceutical preparations have a low enough pyrogen activity to be suitable for use in a human patient, or for veterinary use. In certain embodiments, the pharmaceutical preparation comprises an effective amount of a compound of the invention (e.g., a compound of any one of formulae 1-6 or norfluoxetine enriched for the (R) enantiomer). For example, the present invention provides a pharmaceutical composition comprising a pharmaceutically acceptable excipient and norfluoxetine enriched for the (R) enantiomer in a range of 1 mg to 10 mg. In certain embodiments, the norfluoxetine enriched for the (R) enantiomer is substantially free of (S)-norfluoxetine.

The present invention also relates to a pharmaceutical composition comprising a pharmaceutically acceptable carrier, a compound of the invention (e.g., a compound of any one of formulae 1-6 or norfluoxetine enriched for the (R) enantiomer), and at least one of the following: an agonist of MC4; an allosteric potentiator of MC4; an inhibitor of dopamine reuptake; an inhibitor of norepinephrine reuptake; an inhibitor of both dopamine and norepinephrine reuptake; an MAO-B inhibitor; a dopamine D1 agonist; a dopamine D2 agonist; a dopamine D3 agonist; a dopamine D4 agonist; or a dopamine D5 agonist. In certain embodiments, the pharmaceutical compositions may be for use in treating or preventing obesity, anorexia nervosa, bulimia nervosa, a bulimia-type eating disorder not otherwise specified, metabolic syndrome, or a disorder associate with metabolic syndrome (e.g., obesity, diabetes, hypertension, and hyperlipidemia). In certain embodiments, the pharmaceutical preparations have a low enough pyrogen activity to be suitable for use in a human patient, or for veterinary use.

The present invention further relates to a pharmaceutical composition comprising a pharmaceutically acceptable carrier, a compound of the invention (e.g., a compound of any one of formulae 1-6 or norfluoxetine enriched for the (R) enantiomer), and at least one of the following: bupropion; methylphenidate; sibutramine; sertraline; venlafaxine; atomoxetine; amineptine; benztropine; reboxetine; rasagiline; selegiline; deprenyl; lazabemide; quinpirole; talipexole; sumanirole; bromocriptine; ropinirole; pramipexole; levodopa; amantadine; pergolide; fenoldopam; cabergoline; rotigotine; lysuride; 7-OH DPAT; SKF-38393; apomorphine; or a pharmaceutically acceptable salt, metabolite, or stereoisomer thereof. In certain embodiments, the pharmaceutical compositions may be for use in treating or preventing obesity, anorexia nervosa, bulimia nervosa, a bulimia-type eating disorder not otherwise specified, metabolic syndrome, or a disorder associate with metabolic syndrome (e.g., obesity, diabetes, hypertension, and hyperlipidemia). In certain embodiments, the pharmaceutical preparations have a low enough pyrogen activity to be suitable for use in a human patient, or for veterinary use.

Compounds of the invention (e.g., a compound of any one of formulae 1-6 or norfluoxetine enriched for the (R) enantiomer) may be used in the manufacture of medicaments for the treatment of any diseases disclosed herein.

As used herein, the term “obesity” includes both excess body weight and excess adipose tissue mass in an animal. An obese individual is one having a body mass index of >30 kg/m². While the animal is typically a human, the invention also encompasses the treatment of non-human mammals. The treatment of obesity, as provided in methods of the present invention, contemplates not only the treatment of individuals who are defined as “obese”, but also the treatment of individuals with weight gain that if left untreated may lead to the development of obesity.

Compounds of the invention may have functional antagonist activity versus the CB1 receptor. A “functional antagonist” may be a full antagonist, an inverse agonist, or an allosteric attenuator. Without wishing to be restricted by the proposal, this CB1 activity may mediate the utility of these compounds for the treatment of obesity or eating disorders.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The term “hydrate” as used herein, refers to a compound formed by the union of water with the parent compound:

The term “metabolite” is intended to encompass compounds that are produced by metabolism of the parent compound under normal physiological conditions. For example, an N-methyl group may be cleaved to produce the corresponding N-desmethyl metabolite. Preferred metabolites of the present invention include those that exhibit similar activity to their parent compound (e.g., metabolites that are suitable for the treatment of obesity, anorexia nervosa, bulimia nervosa, or a bulimia-type eating disorder not otherwise specified).

The term “solvate” as used herein, refers to a compound formed by solvation (e.g., a compound formed by the combination of solvent molecules with molecules or ions of the solute).

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

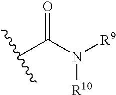

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

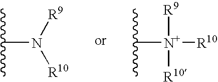

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

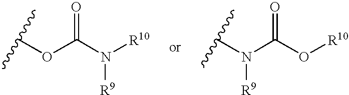

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R⁹,

wherein R⁹ represents a hydrocarbyl group.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —SO₃H, or a pharmaceutically acceptable salt thereof.

The term “sulfonamide” is art-recognized and refers to the group represented by the general formulae

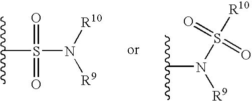

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R⁹, wherein R⁹ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R⁹, wherein R⁹ represents a hydrocarbyl.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

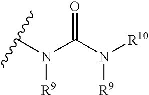

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Methods of preparing substantially isomerically pure compounds are known in the art. If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers. Alternatively, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Techniques for inverting or leaving unchanged a particular stereocenter, and those for resolving mixtures of stereoisomers are well known in the art, and it is well within the ability of one of skill in the art to choose an appropriate method for a particular situation. See, generally, Furniss et al. (eds.), Vogel's Encyclopedia of Practical Organic Chemistry 5^{th} Ed., Longrnan Scientific and Technical Ltd., Essex, 1991, pp. 809-816; and Heller, Acc. Chem. Res. 23: 128 (1990).

The amount of active agent(s) (e.g., a compound of the invention) administered can vary with the patient, the route of administration and the result sought. Optimum dosing regimens for particular patients can be readily determined by one skilled in the art.

Compounds of the invention may be administered to an individual in need thereof. In certain embodiments, the individual is a mammal such as a human, or a non-human mammal. When administered to an individual, the compound of the invention can be administered as a pharmaceutical composition containing, for example, the compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In a preferred embodiment, when such pharmaceutical compositions are for human administration, the aqueous solution is pyrogen free, or substantially pyrogen free, or has low enough pyrogen activity. The excipients can be chosen, for example, to effect delayed release of an agent or to selectively target one or more cells, tissues or organs. The pharmaceutical composition can be in dosage unit form such as tablet, capsule, sprinkle capsule, granule, powder, syrup, suppository, injection or the like. The composition can also be present in a transdermal delivery system, e.g., a skin patch.

The term “low enough pyrogen activity”, with reference to a pharmaceutical preparation, refers to a preparation that does not contain a pyrogen in an amount that would lead to an adverse effect (e.g., irritation, fever, inflammation, diarrhea, respiratory distress, endotoxic shock, etc.) in a subject to which the preparation has been administered. For example, the term is meant to encompass preparations that are free of, or substantially free of, an endotoxin such as, for example, a lipopolysaccharide (LPS).

A pharmaceutically acceptable carrier can contain physiologically acceptable agents that act, for example, to stabilize or to increase the absorption of a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, a compound of the invention. Liposomes, for example, which consist of phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) containing a compound of the invention can be administered to a subject by any of a number of routes of administration including, for example, orally (for example, drenches as in aqueous or non-aqueous solutions or suspensions, tablets, boluses, powders, granules, pastes for application to the tongue); sublingually; anally, rectally or vaginally (for example, as a pessary, cream or foam); parenterally (including intramusclularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin). The compound may also be formulated for inhalation. In certain embodiments a compound of the invention may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein. The most preferred route of administration is the oral route.

The formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal, vaginal, or urethral administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Oreg., U.S.A., 1977).

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, a compound of the present invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can benefit from a combined effect of different therapeutic compounds.

In certain embodiments, a compound of the present invention (e.g., a compound of any one of formulae 1-6 or norfluoxetine enriched for the (R) enantiomer) may be administered conjointly with an agonist of MC4. In certain embodiments, a compound of the present invention (e.g., a compound of any one of formulae 1-6 or norfluoxetine enriched for the (R) enantiomer) may be administered conjointly with an allosteric potentiator of MC4. In certain embodiments, a compound of the present invention (e.g., a compound of any one of formulae 1-6 or norfluoxetine enriched for the (R) enantiomer) may be administered conjointly with an inhibitor of dopamine reuptake. In certain embodiments, a compound of the present invention (e.g., a compound of any one of formulae 1-6 or norfluoxetine enriched for the (R) enantiomer) may be administered cojointly with an inhibitor of norepinephrine reuptake. In certain embodiments, a compound of the present invention (e.g., a compound of any one of formulae 1-6 or norfluoxetine enriched for the (R) enantiomer) may be administered conjointly with an inhibitor of both dopamine and norepinephrine reuptake. In certain embodiments, a compound of the present invention (e.g., a compound of any one of formulae 1-6 or norfluoxetine enriched for the (R) enantiomer) may be administered cojointly with an MAO-B inhibitor. In certain embodiments, a compound of the present invention (e.g., a compound of any one of formulae 1-6 or norfluoxetine enriched for the (R) enantiomer) may be administered cojointly with a dopamine D1 agonist, a dopamine D2 agonist, a dopamine D3 agonist, a dopamine D4 agonist, or a dopamine D5 agonist. Compounds that may be conjointly administered with a compound of the present invention (e.g., a compound of any one of formulae 1-6 or norfluoxetine enriched for the (R) enantiomer) include, but are not limited to, bupropion, methylphenidate, sibutramine, sertraline, venlafaxine, atomoxetine, amineptine, benztropine, reboxetine, rasagiline, selegiline, deprenyl, lazabemide, quinpirole, talipexole, sumanirole, bromocriptine, ropinirole, pramipexole, levodopa (optionally in combination with carbidopa), amantadine, pergolide, fenoldopam, cabergoline, rotigotine, lysuride, 7-OH DPAT, SKF-38393, apomorphine, or a pharmaceutically acceptable salt, metabolite, or stereoisomer thereof.

In certain embodiments, the method of treating prostate cancer comprising administering to a mammal suffering from prostate cancer an effective dose of norfluoxetine (e.g., norfluoxetine enriched for the (R) enantiomer) may comprise administering norfluoxetine (e.g., norfluoxetine enriched for the (R) enantiomer) conjointly with a chemotherapeutic agent. Chemotherapeutic agents that may be conjointly administered with norfluoxetine (e.g., norfluoxetine enriched for the (R) enantiomer) include: aminoglutethimide, amsacrine, anastrozole, asparaginase, bcg, bicalutamide, bleomycin, buserelin, busulfan, camptothecin, capecitabine, carboplatin, carmustine, chlorambucil, cisplatin, cladribine, clodronate, coichicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

Many combination therapies have been developed for the treatment of cancer. In certain embodiments, norfluoxetine (e.g., norfluoxetine enriched for the (R) enantiomer) may be conjointly administered with a combination therapy. Examples of combination therapies with which norfluoxetine (e.g., norfluoxetine enriched for the (R) enantiomer) may be conjointly administered are included in Table 1.

TABLE 1
Exemplary combinatorial therapies for the treatment of cancer.

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, Procarbazine, |
| | Prednisone |
| BEACOPP | Bleomycin, Etoposide, Doxorubicin, Cyclophosphamide, |
| | Vincristine, Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, Prednisone, |
| | Asparaginase |
| CAMP | Cyclophosphamide, Doxorubicin, Methotrexate, |
| | Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or without/ |
| | Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, |
| | Doxorubicin, Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, Prednisone |
| CMFVP | Cyclophosphamide, Methotrexate, Fluorouracil, Vincristine, |
| | Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, Leucovorin, |
| | Cytarabine |
| COMP | Cyclophosphamide, Vincristine, Methotrexate, Prednisone |
| Cooper Regimen | Cyclophosphamide, Methotrexate, Fluorouracil, Vincristine, |
| | Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP(Chronic lymphocytic | Chlorambucil, Prednisone |
| leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, Fluorouracil |
| ICE-T | Ifosfamide, Carboplatin, Etoposide, Paclitaxel, Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, |
| | Melphalan |
| MAC-III | Methotrexate, Leucovorin, Dactinomycin, |
| | Cyclophosphamide |
| MACC | Methotrexate, Doxorubicin, Cyclophosphamide, Lomustine |
| MACOP-B | Methotrexate, Leucovorin, Doxorubicin, Cyclophosphamide, |
| | Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, |
| | Doxorubicin, Bleomycin, Vinblastine |
| MP (multiple myeloma) | Melphalan, Prednisone |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, |
| | Cytarabine, Bleomycin, Vincristine, Methotrexate, |
| | Leucovorin, Cotrimoxazole |
| PRoMACE/MOPP | Prednisone, Doxorubicin, Cyclophosphamide, Etoposide, |
| | Mechlorethamine, Vincristine, Procarbazine, Methotrexate, |
| | Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, |
| | Bleomycin, Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, |
| | Bleomycin |
| VAC | Vincristine, Dactinomycin, Cyclophosphamide |
| VACAdr | Vincristine, Cyclophosphamide, Doxorubicin, Dactinomycin, |
| | Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, |
| | Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or |
| | Mitoxantrone |
| “8 in 1” | Methylprednisolone, Vincristine, Lomustine, Procarbazine, |
| | Hydroxyurea, Cisplatin, Cytarabine, Dacarbazine |

In certain embodiments, norfluoxetine enriched for the (R) enantiomer may be conjointly administered with non-chemical methods of cancer treatment. In certain embodiments, norfluoxetine enriched for the (R) enantiomer may be conjointly administered with radiation therapy. In certain embodiments, norfluoxetine enriched for the (R) enantiomer may be conjointly administered with surgery, with thermoablation, with focused ultrasound therapy, or with cryotherapy.

It is contemplated that a compound of the present invention will be administered to a subject (e.g., a mammal, preferably a human) in a therapeutically effective amount (dose). By “therapeutically effective amount” is meant the concentration of a compound that is sufficient to elicit the desired therapeutic effect (e.g., treatment of obesity or eating disorders). It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

As used herein, compounds of the invention (e.g., compounds of any one of formulae 1-6 or norfluoxetine enriched for the (R) enantiomer) includes the pharmaceutically acceptable salts of compounds of the invention. The pharmaceutically acceptable salts of compounds of the invention can also exist as various solvates, such as with water, methanol, ethanol, dimethylformamide, and the like. Mixtures of such solvates can also be prepared. In general, the solvated forms are equivalent to unsolvated forms and are encompassed within the scope of the present invention. The source of such solvate can be from the solvent of crystallization, inherent in the solvent of preparation or crystallization, or adventitious to such solvent. Certain compounds of the present invention may exist in multiple crystalline or amorphous forms. In general, all physical forms are equivalent for the uses contemplated by the present invention and are intended to be within the scope of the present invention.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, trifluoroacetic, propionic, isobutyric, maleic, malonic, benzoic, succinic, suberic, fumaric, lactic, mandelic, phthalic, benzensulfonic, p-tolylsulfonic, citric, tartaric, methanesulfonic, and the like. Also included are the salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention may contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs form the various salt forms in certain physical properties, such as solubility in polar solvents, but otherwise the salts are equivalent to the parent form of the compound for the purposes of the present invention.

As a particular example, this invention includes the pharmaceutically acceptable acid addition salts of norfluoxetine, such as (R)-norfluoxetine. Since norfluoxetine is an amine, it is basic in nature and accordingly reacts with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, tartaric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, flumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonates, naphthalene-1-sulfonate, naphthalene-2-sulfonate, mandelate, hippurate, gluconate, lactobionate, tartrate, and the like salts. Preferred pharmaceutically acceptable acid addition salts include those formed with mineral acids such as hydrochloric acid and hydrobromic acid, and those formed with organic acids such as fumaric acid, tartaric acid and maleic acid. In certain embodiments, the tartaric acid is (D)-tartaric acid and the resulting salt is the (D)-tartrate salt. In certain embodiments, the pharmaceutically acceptable salt is (R)-norfluoxetine (D)-tartrate.

The pharmaceutically acceptable acid addition salts of norfluoxetine are typically formed by reacting norfluoxetine with an equimolar or excess amount of acid. The reactants are generally combined in a mutual solvent such as diethyl ether or benzene, and the salt normally precipitates out of solution within about one minute to 10 days, and can be isolated by filtration.

Methods of preparing substantially isomerically pure compounds are known in the art. If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers. Alternatively, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Techniques for inverting or leaving unchanged a particular stereocenter, and those for resolving mixtures of stereoisomers are well known in the art, and it is well within the ability of one of skill in the art to choose an appropriate method for a particular situation. See, generally, Furniss et al. (eds.), Vogel's Encyclopedia of Practical Organic Chemistry 5^{th} Ed., Longman Scientific and Technical Ltd., Essex, 1991, pp. 809-816; and Heller, Acc. Chem. Res. 23: 128 (1990).

Norfluoxetine can be prepared by any of a number of methods generally known in the art. For example, there are several methods provided in the literature for making the racemate of norfluoxetine (U.S. Pat. No. 4,313,896). The racemate of norfluoxetine in turn can be resolved, if desired, into its (S) and (R) components by standard methods. In particular, norfluoxetine can be reacted with an enantiomerically pure chiral derivatizing agent, resolved on the basis of the different physicochemical properties of the diastereomeric derivatives, and then converted to the two separate enantiomers of norfluoxetine. One particularly preferred method of accomplishing this derivatization is analogous to that described in Robertson et al., J. Med. Chem., 31, 1412 (1988), wherein fluoxetine was reacted with an optically active form of 1-(1-naphthyl)ethyl isocyanate to form a urea derivative of fluoxetine. A similar mixture of norfluoxetine diastereomeric ureas can be separated through high pressure liquid chromatography into the individual diastereomers. Each individual diastereomer, in turn, can then be hydrolyzed to the individual enantiomers of norfluoxetine.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In certain embodiments, the present invention provides a kit comprising:
a) one or more single dosage forms each comprising a dose of norfluoxetine enriched for the (R) enantiomer in the range of 1 mg to 60 mg;
b) one or more single dosage forms of a chemotherapeutic agent as mentioned above; and
c) instructions for the administration of the norfluoxetine enriched for the (R) enantiomer and the chemotherapeutic agent.

The present invention provides a kit comprising:
a) one or more single dosage forms each comprising a dose of norfluoxetine enriched for the (R) enantiomer in the range of 1 mg to 60 mg and a pharmaceutically acceptable excipient; and
b) instructions for administering the single dosage forms for the treatment of obesity, anorexia nervosa, bulimia nervosa, or a bulimia-type eating disorder not otherwise specified.

The present invention provides a kit comprising:
a) a first pharmaceutical formulation comprising a compound of the invention (e.g., a compound of formulas 1-6 or norfluoxetine enriched for the (R)-enantiomer);
b) a second pharmaceutical formulation comprising at least one of the following: an allosteric potentiator of MC4, an agonist of MC4, an inhibitor of dopamine reuptake, an inhibitor of norepinephrine reuptake, an inhibitor of both dopamine and norepinephrine reuptake, an MAO-B inhibitor, a dopamine D1 agonist, a dopamine D2 agonist, a dopamine D3 agonist, a dopamine D4 agonist, or a dopamine D5 agonist; and
c) instructions for the administration of the first and second pharmaceutical formulations.

The present invention provides a kit comprising:
a) a first pharmaceutical formulation comprising a compound of the invention (e.g., a compound of formulas 1-6 or norfluoxetine enriched for the (R)-enantiomer);
b) a second pharmaceutical formulation comprising at least one of the following: bupropion, methylphenidate, sibutramine, sertraline, venlafaxine, atomoxetine, amineptine, benztropine, reboxetine, rasagiline, selegiline, deprenyl, lazabemide, quinpirole, talipexole, sumanirole, bromocriptine, ropinirole, pramipexole, levodopa (optionally in combination with carbidopa), amantadine, pergolide, fenoldopam, cabergoline, rotigotine, lysuride, 7-OH DPAT, SKF-38393, apomorphine, or a pharmaceutically acceptable salt, metabolite, or stereoisomer thereof; and
c) instructions for the administration of the first and second pharmaceutical formulations.

In certain embodiments, the invention relates to a method for conducting a pharmaceutical business, by manufacturing a formulation or kit as described herein, and marketing to healthcare providers the benefits of using the formulation or kit in the treatment of obesity, anorexia nervosa, bulimia nervosa, a bulimia-type eating disorder not otherwise specified, metabolic syndrome, or a disorder associated with metabolic syndrome (e.g., obesity, diabetes, hypertension, and hyperlipidemia).

In certain embodiments, the invention provides a method for conducting a pharmaceutical business, by providing a distribution network for selling a formulation or kit as described herein, and providing instruction material to patients or physicians for using the formulation to treat obesity, anorexia nervosa, bulimia nervosa, a bulimia-type eating disorder not otherwise specified, metabolic syndrome, or a disorder associated with metabolic syndrome (e.g., obesity, diabetes, hypertension, and hyperlipidemia).

In certain embodiments, the present invention relates to a method for conducting a pharmaceutical business, by providing a distribution network for selling a formulation or kit as described herein, and providing instruction material to patients or physicians for using the formulation to treat obesity, anorexia nervosa, bulimia nervosa, a bulimia-type eating disorder not otherwise specified, metabolic syndrome, or a disorder associated with metabolic syndrome (e.g., obesity, diabetes, hypertension, and hyperlipidemia).

In certain embodiments, the invention comprises a method for conducting a pharmaceutical business, by determining an appropriate formulation and dosage of a compound of the invention (e.g., a compound of formulas 1-6 or norfluoxetine enriched for the (R)-enantiomer) to be administered in the treatment of obesity, anorexia nervosa, bulimia nervosa, or a bulimia-type eating disorder not otherwise specified, conducting therapeutic profiling of identified formulations for efficacy and toxicity in animals, and providing a distribution network for selling an identified preparation as having an acceptable therapeutic profile. In certain embodiments, the method further includes providing a sales group for marketing the preparation to healthcare providers.

In certain embodiments, the invention relates to a method for conducting a pharmaceutical business by determining an appropriate formulation and dosage of a compound of the invention (e.g., a compound of formulas 1-6 or norfluoxetine enriched for the (R)-enantiomer) to be administered in the treatment of obesity, anorexia nervosa, bulimia nervosa, or a bulimia-type eating disorder not otherwise specified, and licensing, to a third party, the rights for further development and sale of the formulation.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Assay Development

cDNA for the G protein GqΔGi chimera was generated by PCR and inserted into the polylinker region of the pcDNA3/hygro+vector (Invitrogen). Stable expression of GqΔGi chimera protein in CHO cell line was generated under hygromycin selection. Human Cannabinoid 1 (CB1) cDNA was inserted into the polylinker region of the pcDNA3.1/(+) vector (Invitrogen) and DNA was introduced into the GqΔGi/CHO cell line by Lipofectamine reagent (Invitrogen). Cell lines stably expressing the CB receptor with Gq ΔGi chimera protein were generated by double selection with hygromycin and G418 (Coward, P., et al. 1999. Chimeric G proteins allow a high-throughput signaling assay of Gi-coupled receptors. Anal Biochem. 270:242-8).

Cell line optimization was done by a proprietary method using our novel automated Direct Sample Injection System (DSIS, ‘Direct mixing and injection for high throughput fluidic systems’ Patent Application Number 20050249635) in conjunction with response. Intracellular Ca²⁺ (Ca²⁺ᵢ) levels were measured with Ca²⁺ chelating fluorescent probes. Here we used the single excitation (UV excitation source), dual emission probe, Indo-1 (Invitrogen/Molecular Probes).

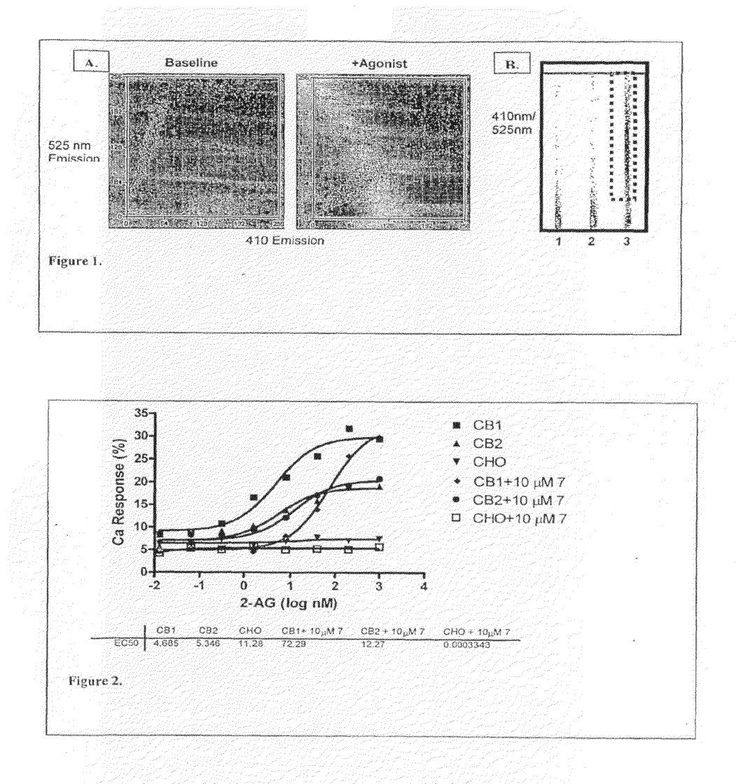

FIG. 1A shows dot plots of cells loaded with Indo-1, displaying low Ca²⁺ᵢ emissions at baseline compared to high Ca²⁺ᵢ emission seen with the addition of agonist. The Indo-1 emissions (410 nm/525 nm) increased and decreased, respectively, as the Ca²⁺ᵢ levels rose and the ratio of 410 nm to 525 nm emissions provided a stable index of Ca²⁺ᵢ, fundamentally independent of the extent of dye loading. We exploited the intrinsic heterogeneity of individual cells within a cell population of a single cell line searching for the desired functional assay response by imposing it as the selection criterion. This cellular evolution approach was used to select the subset of cells that couple the transfected GPCR protein to a desired functional assay readout. The oval represents the type of gate that can be set as a sorting criteria. FIG. 1B is a dot plate displaying cells as run through DSIS with the Indo-1 emissions displayed as a ratio (y-axis) over time (x-axis). Here we sorted the CB1 cells that responded to 2-AG with an increase in Ca²⁺ᵢ. Samples 1 and 2 are at baseline levels while 3 displays the response seen in the presence of agonist (dashed rectangle).

CB1 Cells were plated 24-48 hours in advance, and were harvested with trypsin at ˜80% confluency. Cells were then centrifuged and resuspended two times in Hybridoma Media, the final time at a concentration of 1×10⁶ cells/mL. Two μM of Indo-1 were added and cells were incubated for 1 hour on a rotator at room temperature. Cells were washed two times and resuspended at a concentration of 2×10⁶ cells/mL in Hybridoma Media. The CB1 agonist, 2-Arachidonylgylcerol (2-AG, Tocris #1298), was prepared at a concentration which was 4 times the Emax concentration for the CB1 cells. Twenty μL of 2-AG were placed in 8 rows of a 384-well plate. The probe-loaded cells were then placed in the cell suspension system on the DSIS where they were continuously rocked to keep them in a suspended state. The 2-AG plate was also transferred to the DSIS-FACS.

To run the sorting assay, DSIS added 60 μL of cells to one well of 2-AG in the 384-well plate. This was done at an injection rate of 40 μL/second, which mixed the cells with the compound. The sample was then injected into a MoFlo cytometer (Dako-Cytomation). Using Summit software, dot plots that display the ratio of the 410 nm and 525 nm emissions of the Indo-1 probe were used to set a gate for cells displaying a high Ca²⁺ᵢ response. Cells were injected into the cytometer for 45 seconds each round. This process continued iteratively until all cells were sorted. Cells passing the sort criteria were deflected into a 5 mL collection tube containing 2 mL of FBS. Once the sort was complete, the cells were transferred into a new tissue culture flask and the sorted population was expanded. The new, sorted CB1 population was then prepared for testing, loaded with Indo-1 and analyzed for Ca²⁺ᵢ response. The complete sorting procedure was repeated until a cell line was developed that had a response rate greater than 70%. For the CB1 cell line, the initial response rate was 12% and after 3 sorts increased to 75% of the cells. In addition to collecting a population of sorted cells, the Cyclone adaptor to the MoFlo cytometer sorted a single cell into each well of a 96-well plate for clonal sorting. The resulting individual clonal populations were then assayed for Ca²⁺ᵢ response. One clonal CB1 population with a Ca²⁺ᵢ response rate of 80% was chosen for subsequent screening assays.

### CB1 Allosteric Modulator Screening:

The screening process assayed both the CB1 (clonal) expressing cell line and the Control cell line (CHO GqΔGi cells) simultaneously. This was done by staining one population with a tracker dye. The system used herein consisted of an initial treatment with Biotin-X DHPE (Invitrogen/Molecular Probes), a phospholipid conjugated to biotin. The phospholipid portion inserted into the cell membrane leaving the biotin exposed on the cell surface. This was followed by a secondary treatment with an Alexa dye conjugated to streptavidin. The populations were then distinguished by their respective fluorescent signatures.

The CB1 and Control Cells were plated 24-48 hours in advance, and were harvested with trypsin at ˜80% confluency. Cells were then centrifuged and resuspended two times in Hybridoma Media, the final time at a concentration of 1×10⁶ cells/mL. Both cell lines were then loaded with 2 μM of Indo-1 plus 3 μg/mL Biotin-X DHPE and then were incubated for 1 hour on a rotator at room temperature. Cells were washed two times and resuspended at a concentration of 1×10⁶ cells/mL in Hybridoma Media. The CB1 cell line then received 2 μg/ml of Alexa 488-streptavidin (Invitrogen/Molecular Probes). Cells were incubated for an additional 30 minutes on a rocker at room temperature. Both cell lines were centrifuged and washed 2 times in Hybridoma Media with the final resuspension at 5×10⁵ cells/mL.

The Novasite Library of compounds was set up in 96-well V-bottom plates (Falcon). Each plate held 80 compounds located in columns 2-11. Compounds were initially solubilized in DMSO, and then were diluted with PBS. The final assay plates had 20 μL/well of 50 μM compound (in PBS+1% DMSO). Columns 1 and 12 contain PBS+1% DMSO and were used as Background and Control wells.

The probe-loaded CB1 and Control cell mixture was placed in the Cell Suspension System on the DSIS where they were continuously rocked to keep them in a suspended state. To screen for allosteric modulators, we used an EC₅₀ concentration of a natural ligand of the receptor as a control response. Each day a new aliquot of 2-AG was used to prepare a dose/response determination plate. Ten 2-AG concentrations were used starting at 30 μM, then diluted at half log intervals down to 1 nM. They were in the plate at 5× these concentrations. For this initial determination, DSIS was set to agonist mode. Screening assays were run on the CyAn Cytometer (Dako-Cytomation). DSIS added 80 μL of cells to the first well of the dose/response plate, the mixture incubated for 13 seconds, then was injected into the CyAn. This was repeated for each well. The DSIS software, NVS Sampler, recorded a timing file and the CyAn software, Summit, recorded a data file. These two files were then compiled and analyzed by our proprietary software, NVS Analyzer, to determine the percent of cells that responded to each concentration. The data was transferred to GraphPAD Prism for a non-linear regression curve fit that determined the EC₅₀. To set up the allosteric screen, 5 mL of 2-AG was prepared, from the same aliquot used for the dose/response assay, at a concentration 5 times the EC₅₀.

The allosteric screening assay was conducted with DSIS set in antagonist mode with preincubation. The cells were in place, the 2-AG (5×EC₅₀) was added to the appropriate vial holder and the first compound plate was in place. Each plate was run in two segments, rows 1-4 then rows 5-8. Each plate and segment had an individual code that was entered at the start of each run. The parameters of this screen included a 2 minute incubation after 60 μL of cells were added to the compound well. The 2-AG, 20 μL, was then added to the well and there was another 13 second incubation. The cell mixture was then injected into the CyAn for a 45 second interrogation. Wells in column one had no compound and did not receive agonist. This was our background or baseline measurement. Wells in column 12 had no compound, but received the EC₅₀ concentration of 2-AG. These were the control wells that were used to determine if a compound had a potentiating or attenuating effect. Subsequent plates were screened accordingly.

### Analysis:

When the screen was complete, the data files and the timing files were analyzed using NVS Analyzer. Once the data was compiled, it was exported into our ActiviyBase database system and the SARgen query tool was used for final analysis, hit detection and formatting. An average response for the control wells in each plate segment was determined, and the compound wells in that segment were compared to that average. A compound that elicited a response that was more than 25% plus or minus the average of the control, was determined a hit. Any compound that affected the control cell line was deleted from the list.

### Validation:

Compounds determined as hits from the primary screen were cherry picked into new plates for a secondary screen. This was done using DSIS in agonist mode as outlined above in the EC₅₀ determination. Control and CB1 cells were prepared using the same protocol as used for the primary screening assay. DSIS added the cell suspension to the wells then transferred the mixture directly to the CyAn for analysis. This was done to determine if the perceived potentiation or attenuation was due to agonist activity of the compound. As we were looking for an allosteric attenuator or antagonist for the CB1 receptor, our hit list was comprised of those compounds that displayed an attenuating effect in the primary screen and lacked an agonist response in the secondary screen. These compounds were then used in dose-response experiments. 2-AG dose-response determination plates were prepared as outlined above and were run with or without the compounds of interest. FIG. 2 provides the dose response curves of CB1, CB2 and CHO control cells to 2-AG with or without compound 7. Compound 7 induced a right shift in the 2-AG response for CB1, indicative of an allosteric attenuator or an antagonist. No effect was seen for CB2 receptor bearing cells. At this point, compound 7 was selected for further validation as the data displayed potential attenuator/antagonist activity and selectivity for the CB1 receptor over the CB2 receptor.

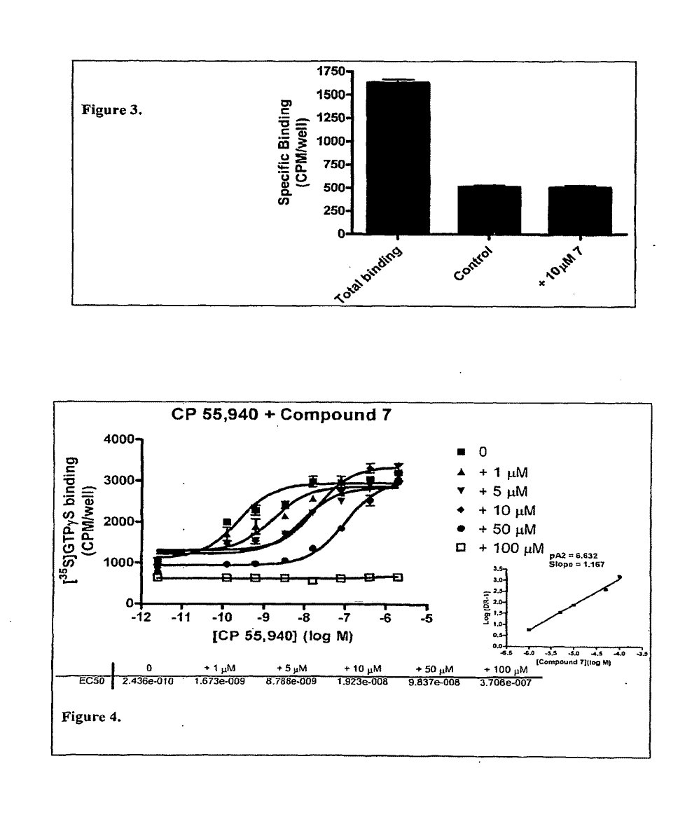

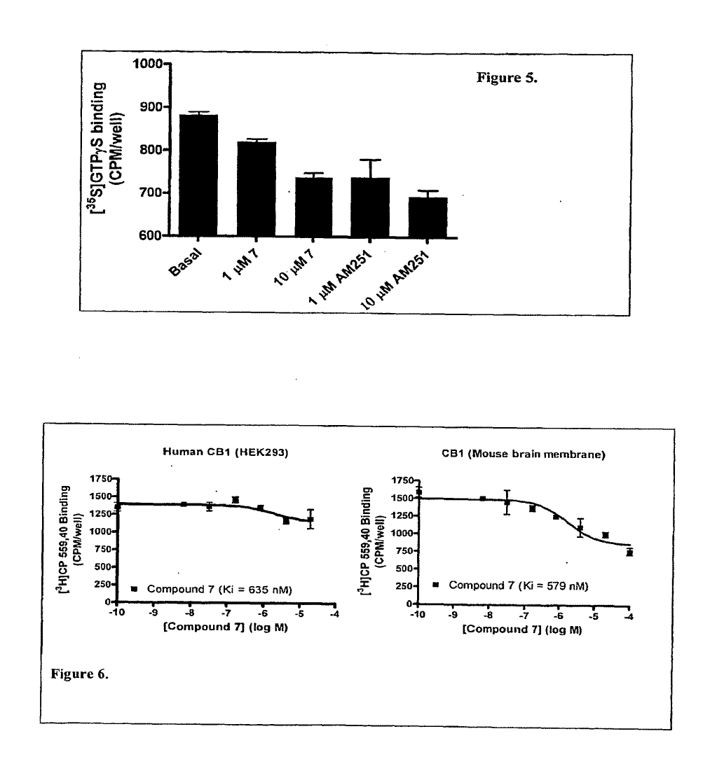

Dissociation kinetics assays were performed with the non-selective cannabinoid receptor agonist ([³H]CP 55,940) (0.75 nM) in the binding buffer containing 50 mM Tris-HCl, pH 7.4, 3 mM MgCl₂, 1 mM EDTA, 0.2% BSA using CHO-k1 cell membranes stably expressing human CB1 receptors in 96-well plate format. The CB1 receptor membranes (10 μg/well) were incubated with 0.75 nM [³H]CP 55,940 in 100 μl binding buffer at room temperature for 2 h. Dissociation was initiated with addition of 100 μl unlabeled CP 55,940 (10 μM) in binding buffer in the absence or presence of different concentrations of compounds (compound 7). Dissociation was carried out at room temperature for indicated time. To determine the non-specific binding, experiments were also performed in the presence of 10 μM unlabeled CP 55,940. Binding was terminated by addition of cold binding buffer and filtrated on Whatman GF/B glass-fiber filters using a sampling manifold. The filters were washed 6 times with cold binding buffer and air-dried overnight. The radioactivity was quantitated on a TopCounter (PerkinElmer) after adding scintillation fluid. Specific binding was defined as the difference between the binding in the presence and absence of 10 μM unlabeled CP 55,940. FIG. 3 shows that compound 7 had no effect on the dissociation rate of [³H]CP 55,940. This indicated that the compound is either an antagonist or an inverse agonist, but not an allosteric modulator.

[³⁵S]GTPγS binding assays were performed with CHO-k1 cell membranes stably expressing human CB1 receptors in 96-well ScintiPlate (PerkinElmer). The membranes (12.5 μg/well) were preincubated with compound 7 in the binding buffer (50 mM Tris-HCl, pH 7.4, 100 mM NaCl, 3 mM MgCl₂, 0.2 mM EDTA, 0.2% BSA and 10 μM GDP) at 30° C. for 30 min, then added various concentrations of agonists (WIN 55212-2 or CP 55,940) and [³⁵S]GTPγS (0.2 nM) in a final volume of 200 μl. Reactions were carried out at 30° C. for 60 min, followed by centrifugation of plate at 37000 rpm for 15 min. After removing supernatant, the radioactivity was quantitated on a TopCounter. Data were analyzed by non-linear regression using program GraphPAD Prism and are shown in FIGS. 4 & 5. FIG. 4 shows that a pA2 estimation of compound 7 using CP 55940 as an agonist with the CB1 cell line also did not support an allosteric effect against the agonist. FIG. 5 shows the results of testing between antagonist activity and inverse agonist activity by subjecting compound 7 to a [³⁵S]GTPγS binding assay. The results indicate that compound 7 displayed inverse agonist activity, dropping the response below basal levels.

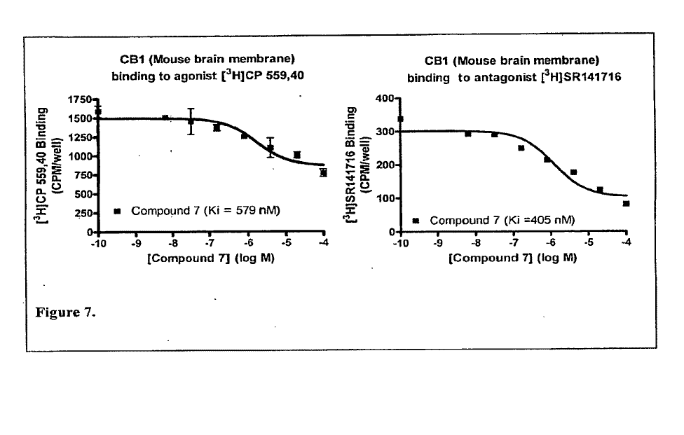

Competition ligand binding assays were performed with the non-selective cannabinoid receptor agonist ([³H]CP 55,940) (0.75 nM) in the binding buffer containing 50 mM Tris-HCl, pH 7.4, 3 mM MgCl₂, 1 mM EDTA, 0.2% BSA using CHO-k1 cell membranes stably expressing human CB1 receptors or mouse brain membrane preparations in 96-well plate format. The receptor membranes (10 μg/well) were pre-incubated with different concentrations of tested compounds in the binding buffer at room temperature for 30 min prior to addition of 0.75 nM [³H]CP 55,940 in a final volume of 100 μl. The binding was carried out at room temperature for another 2 h. To determine the non-specific binding, experiments were also performed in the presence of 10 μM unlabeled CP 55,940. Binding was terminated by addition of cold binding buffer and filtrated on Whatman GF/B glass-fiber filters using a sampling manifold. The filters were washed 6 times with cold binding buffer and air-dried overnight. The radioactivity was quantitated on a TopCounter (PerkinElmer) after adding scintillation fluid. Specific binding was defined as the difference between the binding in the presence and absence of 10 μM unlabeled CP 55, 940. Data were analyzed by non-linear regression using program GraphPAD Prism and are shown in FIGS. 6 & 7. FIG. 6 shows that compound 7 displayed differential binding properties to human and mouse CB1 receptors. Binding of [³H]CP 55,940 was partially inhibited on human CB1 cell membranes but was almost fully inhibited in mouse brain membranes. FIG. 7 shows that compound 7 inhibited both agonist (CP 55940) and antagonist/inverse agonist (SR 141716) binding to mouse CB1 (mouse brain membrane).

### Example 2

### Effect of Treatment X and Treatment Y on the Body Weight, Food and Water Intake of Male C57BL/6J Mice which Exhibit Diet Induced Obesity

The goal of this study was to investigate whether repeated administration of Treatment X, a 1:1 w:w combination of R-norfluoxetine hydrochloride and bupropion hydrochloride, and Treatment Y, a combination of R-norfluoxetine and an MC4 allosteric potentiator, alters the body weight and daily food and water intake in C57BL/6J mice exhibiting obesity due to access to a high fat diet. Sibutramine, which is currently used clinically, and rimonabant, which has recently received regulatory approval for the management of obesity were used as reference compounds.

### Animals:

Sixty-five C57BL/6J mice (7-8 weeks of age) were ordered from Charles River, Margate, Kent. Mice were group housed in polypropylene cages with free access to a high fat diet (D12451 45% of Kcal derived from fat; Research Diets, New Jersey, USA) and tap water at all times. Animals were maintained at 21±4° C. and 55±20% humidity on a normal phase 12 h light-dark cycle (lights on 04:30 h)

### Experimental Procedures:

Animals were exposed to the high fat diet for 16 weeks. During this time body weight was recorded weekly. At the end of 14 weeks animals were singly housed in polypropylene cages for a further two week period (weeks 14-16) and placed on reverse phase lighting (lights off for 8 h from 9.30-17.30 h) during which time the room was illuminated by red light. Animals were dosed with vehicle orally throughout the baseline period. Body weight and food and water intake was recorded daily. Towards the end of the baseline period animals were allocated to one of seven groups (see table 2 below). Upon completion of the baseline period, mice were dosed for 28 days with vehicle or test drug as described below.

TABLE 2

| Group | Treatment | n |
|---|---|---|
| A | Vehicle | 11 |
| B | Treatment X (20 mg/kg po) | 9 |
| C | Treatment X (40 mg/kg po) | 9 |
| D | Treatment Y (20 mg/kg po) | 9 |
| E | Treatment Y (40 mg/kg po) | 9 |
| F | Sibutramine (20 mg/kg po) | 9 |
| G | Rimonabant (10 mg/kg po) | 9 |

Body weight and food and water intake were recorded daily. Following drug administration the animals were examined and any overt behaviour was recorded. For all dosing, the morning session was timed such that approximately half the mice were dosed at the time of lights out (09:30).

### Drugs:

The test compounds were dissolved in 1% methylcellulose. Drugs were made up fresh each day 1-2 h before dosing and were administered using a dose volume in the range of 1-3 ml/kg. Drug doses were expressed as free base.

### Data and Statistical Analysis:

Resulting body weights, food intake and water intake were expressed as mean values±SEM, and the SEMs are calculated from residuals of the statistical model. Body weight data was analysed by ANCOVA with Day 1 as covariate followed by appropriate comparisons (two-tailed) to determine significant differences from the control group. P<0.05 was considered to be statistically significant. Daily food and water intake data was analysed by ANOVA.

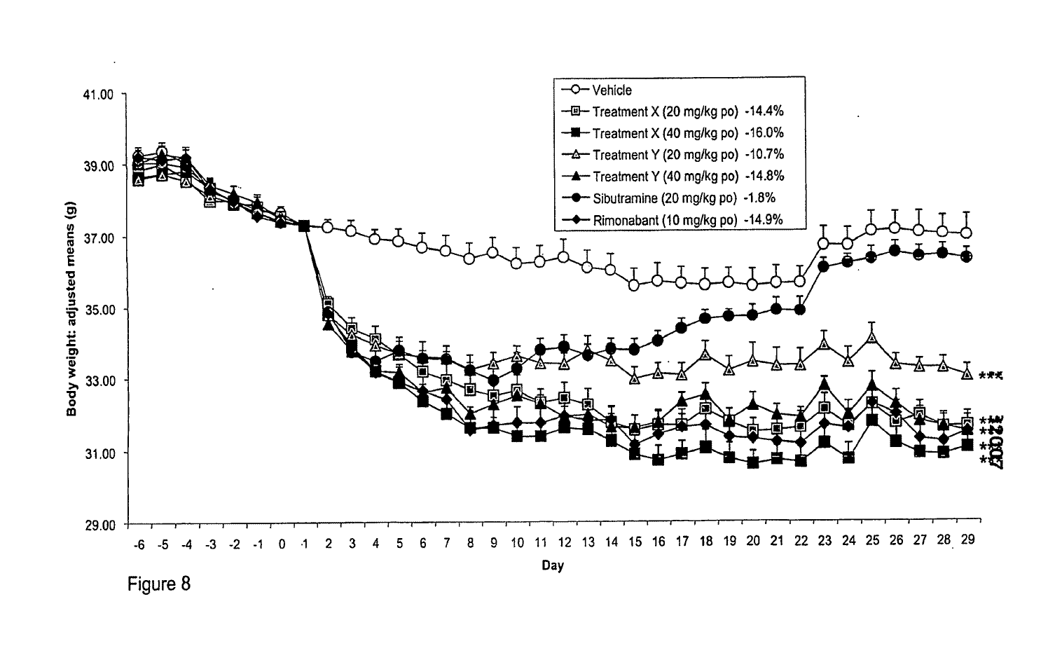

FIG. 8 shows the results of oral administration of Treatment X, Treatment Y, Sibutramine and Rimonabant on the body weight of diet-induced obese male C57BL/6J mice. Drug treatment commenced on Day 1. Treatment X, Treatment Y, and Rimonabant all demonstrated statistically significant weight reduction as compared to vehicle on day 29 as assessed using Dunnett's test (p<0.001). Administration of Treatment X at 20 mg/kg and 40 mg/kg resulted in a 14 and 16% reduction of body weight respectively. This compares to only a 2% reduction in body weight for sibutramine administered at 20 mg/kg, and is comparable to the 15% reduction in body weight for rimonabant administered at 10 mg/kg. Administration of Treatment Y at 20 mg/kg and 40 mg/kg resulted in an II and 14% reduction of body weight respectively.

### Example 3

### Effect of Acute Administration of Test Compounds on Mouse Food Intake

The goal of this study was to investigate the effects of various ratios of bupropion:(R)-norfluoxetine on body weight and food and water intake in male C57BL/6J mice habituated to the daily presentation of a palatable wet mash diet. Rimonabant was used as a reference compound. Animals were maintained on normal-phase lighting. Test compounds were administered orally and measurements were made over the following 24 hours. All experiments included appropriate vehicle-treated control groups.

### Materials and Methods:

Sixty-two male C57BL/6J mice (weight range 20-25 g) were ordered from Harlan UK, Bicester, UK. The mice were individually housed in polypropylene cages at a temperature of 21±4° C. and 55%±20% humidity. Animals were maintained on a normal phase light-dark cycle (lights off for 12 h from 19:00-07:00 h) during which time the room was illuminated by red light. Animals had free access to a standard pelleted rodent diet and tap water at all times. In addition, animals were habituated to a daily presentation of a wet mash diet (1 part powdered chow:1.5 parts tap water) placed on a dish on the cage floor. Animals were maintained under these conditions for at least ten days before experimentation commenced.

### Experimental Procedures:

Typically an acute study was run each week with each study containing 60 animals. On the day prior to testing, the experimental animals were randomly allocated to suitable treatment groups. Animals were weighed and 2 h wet mash intake was calculated (to the nearest 0.1 g). Simple in-house data and statistical analysis was performed to investigate whether there were any significant differences between the treatment groups at baseline. Animals were reallocated into different groups if necessary to resolve any significant differences and ensure that the groups were balanced before drug treatment.

On the test day, animals were briefly removed from the home cage, weighed and dosed with either vehicle, test compound, or a positive control. The table below describes the various treatment groups for a typical two-week period of the study.

TABLE 3
Week 1

| Group | Treatment | n |
|---|---|---|
| A | Vehicle (po) | 10 |
| B | Dose A of first test compound | 10 |
| C | Dose B of first test compound | 10 |
| D | Dose C of first test compound | 10 |
| E | Dose D of first test compound | 10 |
| F | Positive control | 10 |

TABLE 4
Week 2

| Group | Treatment | n |
|---|---|---|
| A | Vehicle (po) | 10 |
| B | Dose A of second test compound | 10 |
| C | Dose B of second test compound | 10 |
| D | Dose C of second test compound | 10 |
| E | Dose D of second test compound | 10 |
| F | Positive control | 10 |

Where the top dose of the test compound had not been tested in vivo before, then a pilot study using two mice was initially undertaken to test that the compound was tolerated.

Drug administration occurred 60 minutes before the presentation of ‘wet mash’ which was at approximately 09:30 am. Food pellets were removed and water bottles were weighed (to the nearest 0.1 g) at the time of drug administration. Wet mash and the water bottle were weighed 1, 2, and 4 h after presentation. The mash was replaced with a fresh quantity of wet mash at the 4 hour time point. Wet mash and water bottle weights were re-weighed at the 6 hour time point. Wet mash was replaced with a known quantity of standard pellets at the 6 hour time point. Food pellets, water bottles, and animals were also weighed 24 h post dosing. Food and water intakes of the different groups of animals were measured concurrently. The animals were monitored at each reading and any overt drug-induced behavioural effects were recorded. Animals were randomised into different treatment groups and could be re-used (up to 8 times in total) following a wash-out period of at least 6 drug-free days between different treatments.

### Drugs:

All drug solutions were given in a dose volume of 3 ml/kg.

Treatment A was a 10:1 weight ratio of bupropion hydrochloride:(R)-norfluoxetine (D)-tartrate.

Treatment B was a 1:4 weight ratio of bupropion hydrochloride:(R)-norfluoxetine (D)-tartrate.

Treatment C was a 1:6 weight ratio of bupropion hydrochloride:(R)-norfluoxetine (D)-tartrate.

Treatment D was a 1:10 weight ratio of bupropion hydrochloride:(R)-norfluoxetine (D)-tartrate.

### Data and Statistical Analysis:

Results (body weights (g) at 0, 24 h; change in body weight (g) over 24; food and water intake at 1, 2, 4, 6 and 24 h and between 1-2 h, 2-4 h, 4-6 h) were expressed as mean values±SEM. Food and water intake was expressed in g. The exact statistical methods employed depended on the data obtained; however, statistical comparisons between the food and water intakes and body weights of different groups of mice were usually made by analysis of variance followed by multiple comparisons tests (two-tailed). P<0.05 was considered to be statistically significant.

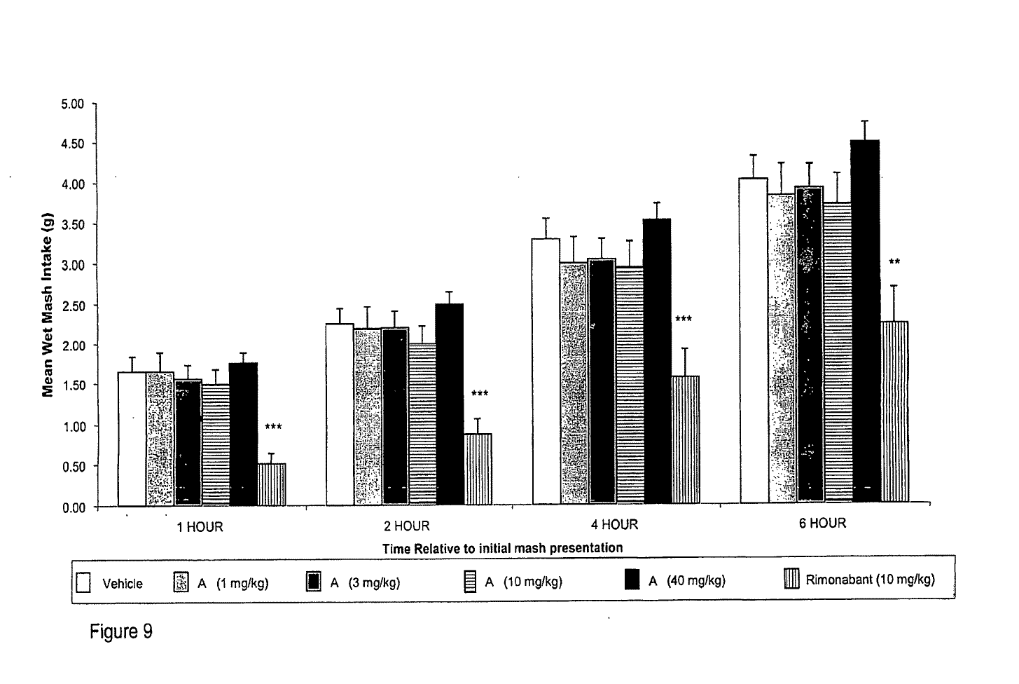

FIG. 9 shows the effect of Treatment A, dosed orally at 1, 3, 10 and 40 mg/kg, and rimonabant, dosed orally at 10 mg/kg, on the consumption of wet mash in lean male C57BL/6J mice (n=10). Data was analyzed by one-way ANOVA and Dunnett's test to assess differences versus the control group. ** indicates p<0.01; and *** indicates P<0.001.

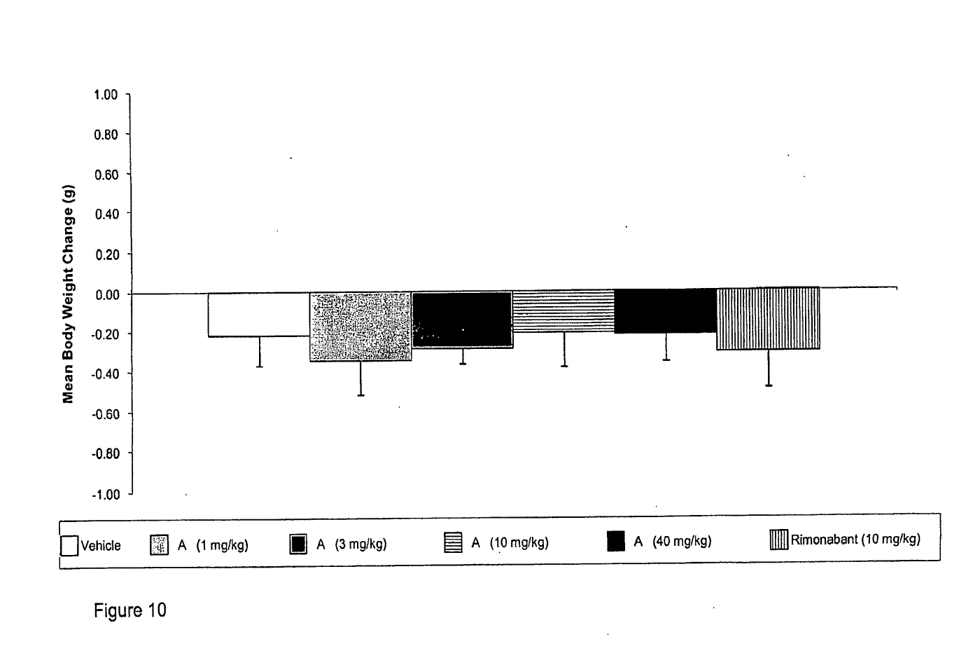

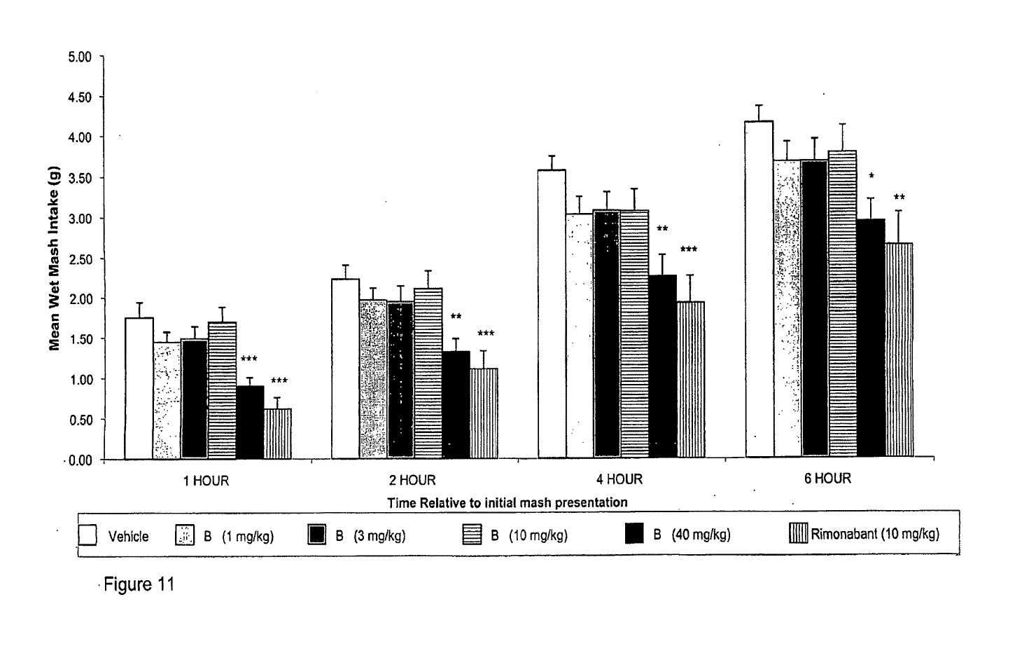

FIG. 10 shows the effect of Treatment A, dosed orally at 1, 3, 10 and 40 mg/kg, and rimonabant, dosed orally at 10 mg/kg, on 24 hour body weight change in lean male C57BL/6J mice (n=9-10). Data was analyzed by one-way ANOVA. No significant differences versus vehicle were observed.

FIG. 11 shows the effect of Treatment B, dosed orally at 1, 3, 10 and 40 mg/kg, and rimonabant, dosed orally at 10 mg/kg, on the consumption of wet mash in lean male C57BL/6J mice (n=9-10). Data was analyzed by one-way ANOVA and Dunnett's test to assess differences versus the control group. * indicates p<0.05; ** indicates p<0.01; and *** indicates P<0.001. Treatment B significantly reduced wet mash intake when dosed orally at 40 mg/kg. At least a 25% reduction in mean wet mash intake as compared to vehicle was observed at the 1, 2, and 4 hour timepoints when treatment B was dosed orally at 40 mg/kg.

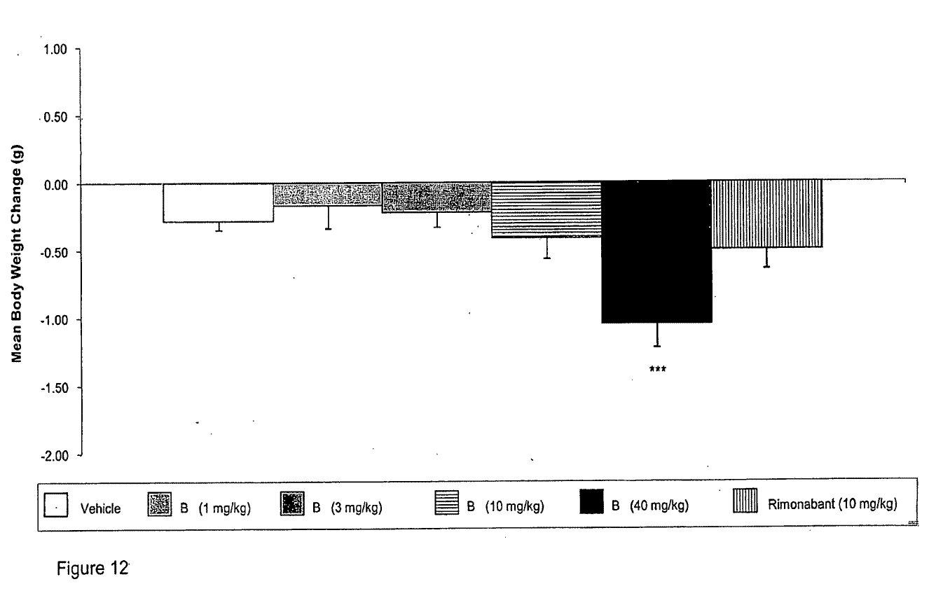

FIG. 12 shows the effect of Treatment B, dosed orally at 1, 3, 10 and 40 mg/kg, and rimonabant, dosed orally at 10 mg/kg, on 24 hour body weight change in lean male C57BL/6J mice (n=10). Data was analyzed by one-way ANOVA and Dunnett's test to assess differences versus the control group. *** indicates P<0.001. Treatment B significantly reduced 24 hour body weight gain when dosed orally at 40 mg/kg. Treatment B, dosed orally at 40 mg/kg, reduced 24 hour body weight gain by at least 200% more than vehicle.

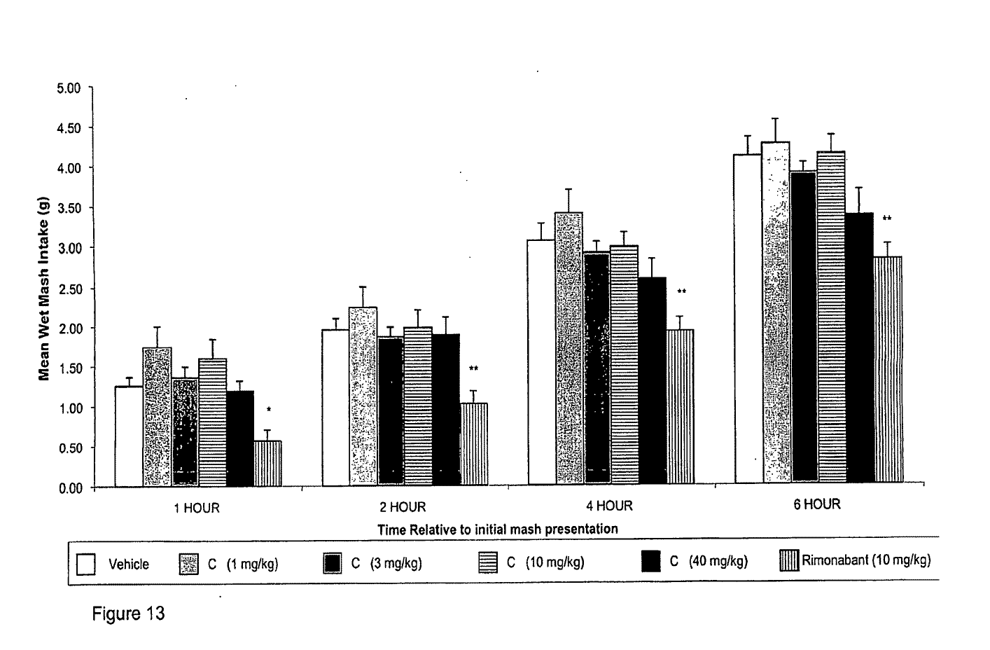

FIG. 13 shows the effect of Treatment C, dosed orally at 1, 3, 10 and 40 mg/kg, and rimonabant, dosed orally at 10 mg/kg, on the consumption of wet mash in lean male C57BL/6J mice (n=9-10). Data was analyzed by one-way ANOVA and Dunnett's test to assess differences versus the control group. * indicates p<0.05; and ** indicates p<0.01. Treatment C did not result in a significant observed effect on wet mash intake.

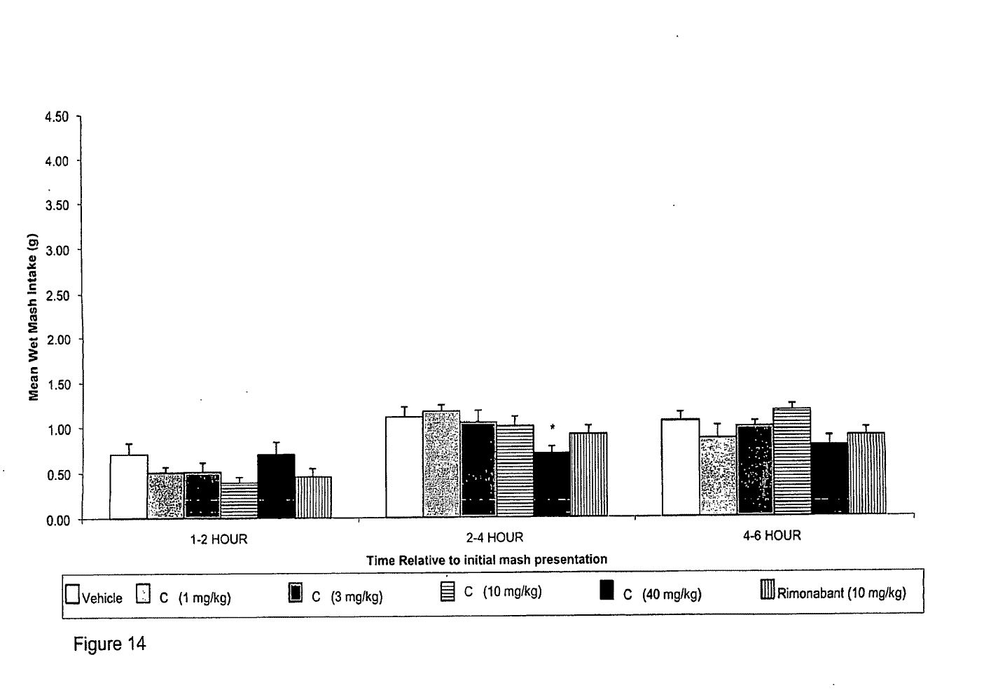

FIG. 14 shows interval data for the effect of Treatment C, dosed orally at 1, 3, 10 and 40 mg/kg, and rimonabant, dosed orally at 10 mg/kg, on the consumption of wet mash in lean male C57BL/6J mice (n=9-10). Data was analyzed by one-way ANOVA and Dunnett's test to assess differences versus the control group. * indicates p<0.05.

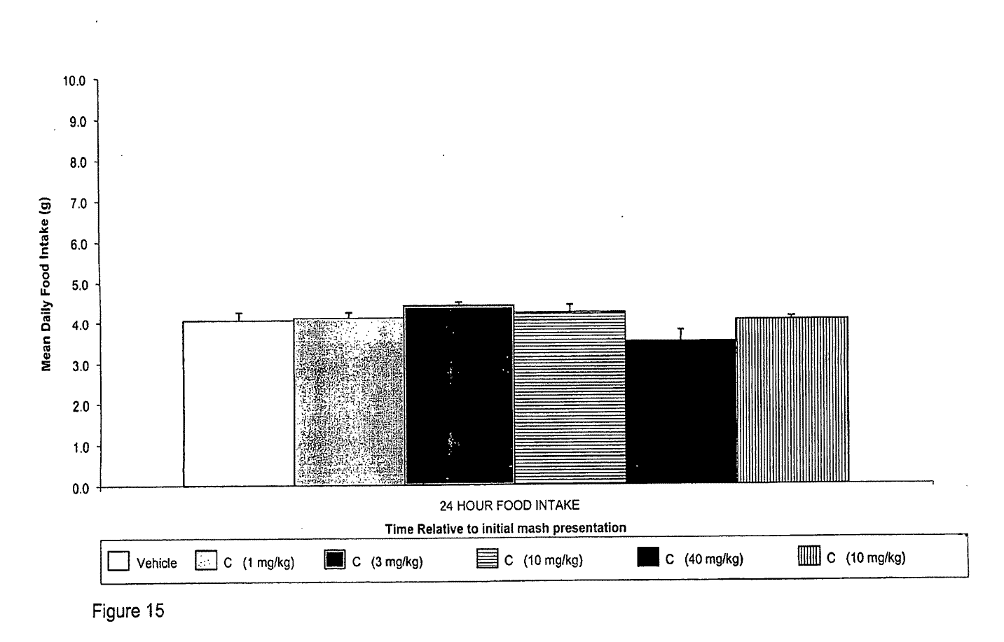

FIG. 15 shows the effect of Treatment C, dosed orally at 1, 3, 10 and 40 mg/kg, and rimonabant, dosed orally at 10 mg/kg, on daily food intake of lean male C57BL/6J mice (n=9-10). Data was analyzed by one-way ANOVA. No significant differences versus vehicle were observed.

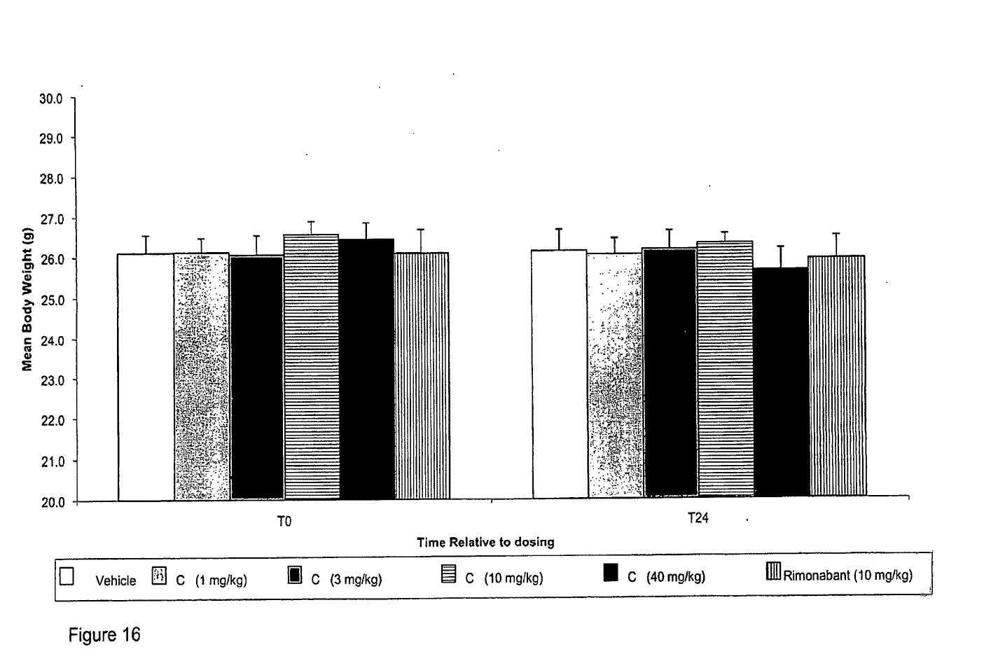

FIG. 16 shows the effect of Treatment C, dosed orally at 1, 3, 10 and 40 mg/kg, and rimonabant, dosed orally at 10 mg/kg, on the body weight of lean male C57BL/6J mice (n=9-10). Data was analyzed by one-way ANOVA. No significant differences versus vehicle were observed.

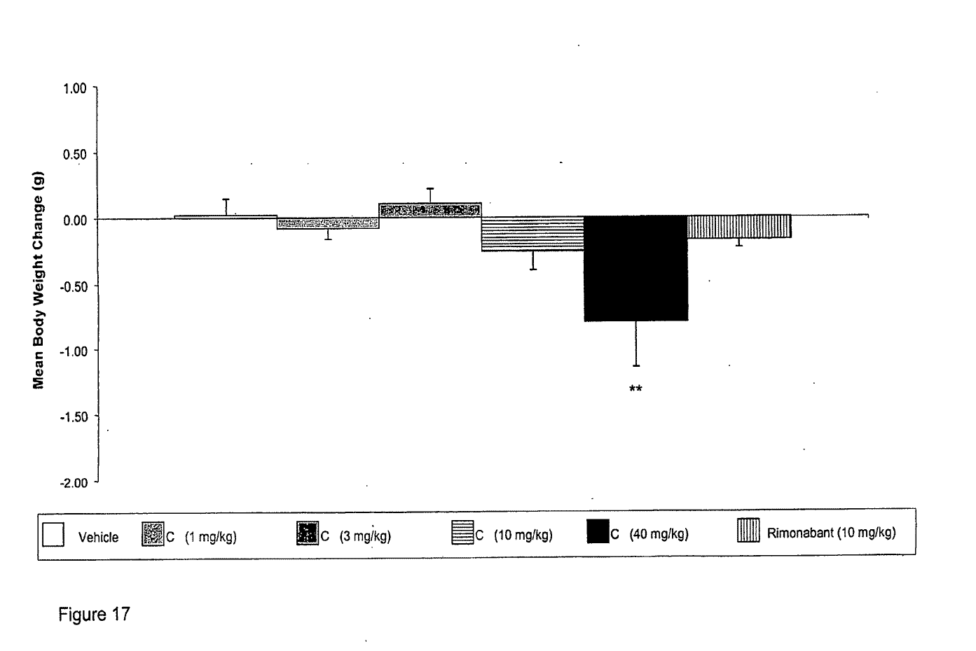

FIG. 17 shows the effect of Treatment C, dosed orally at 1, 3, 10 and 40 mg/kg, and rimonabant, dosed orally at 10 mg/kg, on 24 hour body weight change in lean male C57BL/6J mice (n=9-10). Data was analyzed by one-way ANOVA and Dunnett's test to assess differences versus the control group. ** indicates P<0.01. Treatment C significantly reduced 24 hour body weight gain when dosed orally at 40 mg/kg. Treatment C, when dosed orally at 40 mg/kg, showed a loss of at least 0.5 g mean body weight as compared to essentially no change in body weight with vehicle.

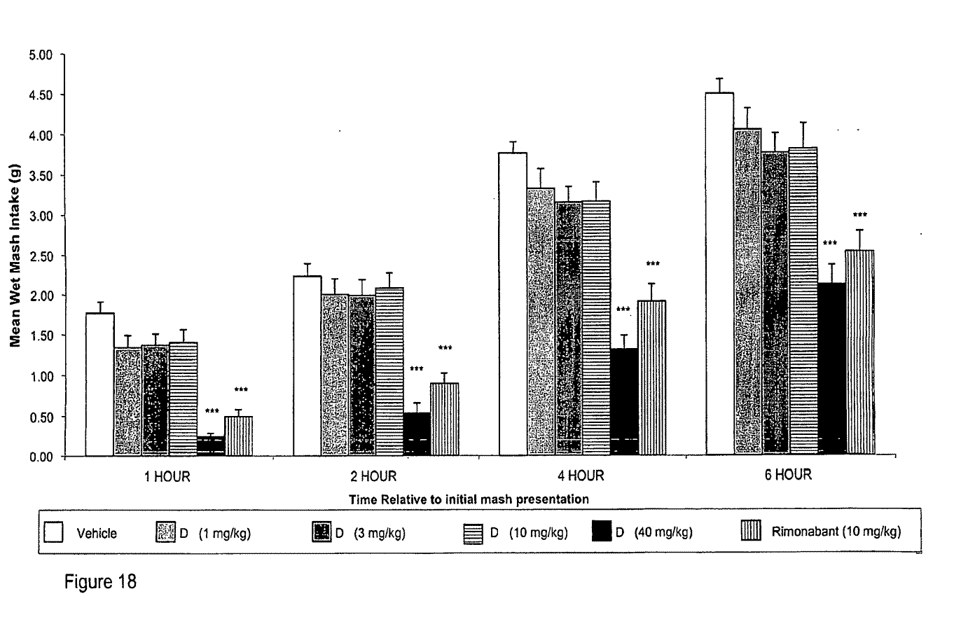

FIG. 18 shows the effect of Treatment D, dosed orally at 1, 3, 10 and 40 mg/kg, and rimonabant, dosed orally at 10 mg/kg, on the consumption of wet mash in lean male C57BL/6J mice (n=10). Data was analyzed by one-way ANOVA and Dunnett's test to assess differences versus the control group. *** indicates p<0.001. Treatment D significantly reduced the consumption of wet mash when dosed orally at 40 mg/kg. At least a 50% reduction in mean wet mash intake as compared to vehicle was observed at the 1, 2, and 4 hour timepoints when treatment D was dosed orally at 40 mg/kg. At least a 30% reduction in mean wet mash intake as compared to vehicle was observed at the 6 hour timepoint when treatment D was dosed orally at 40 mg/kg.

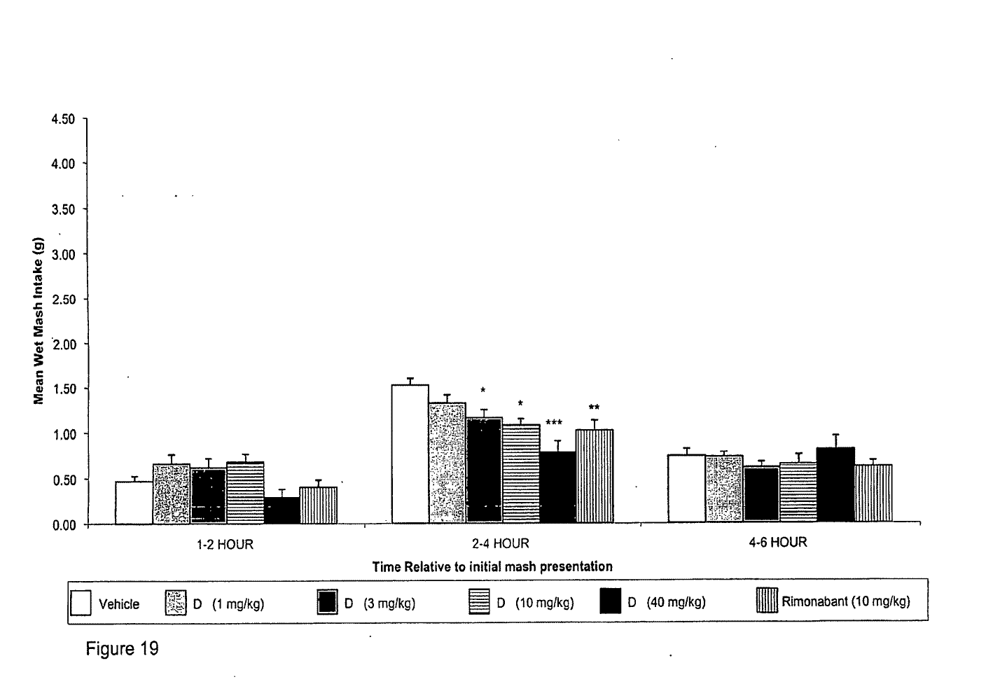

FIG. 19 shows interval data for the effect of Treatment D, dosed orally at 1, 3, 10 and 40 mg/kg, and rimonabant, dosed orally at 10 mg/kg, on the consumption of wet mash in lean male C57BL/6J mice (n=10). Data was analyzed by one-way ANOVA and Dunnett's test to assess differences versus the control group. * indicates p<0.05; ** indicates p<0.01; and *** indicates P<0.001.

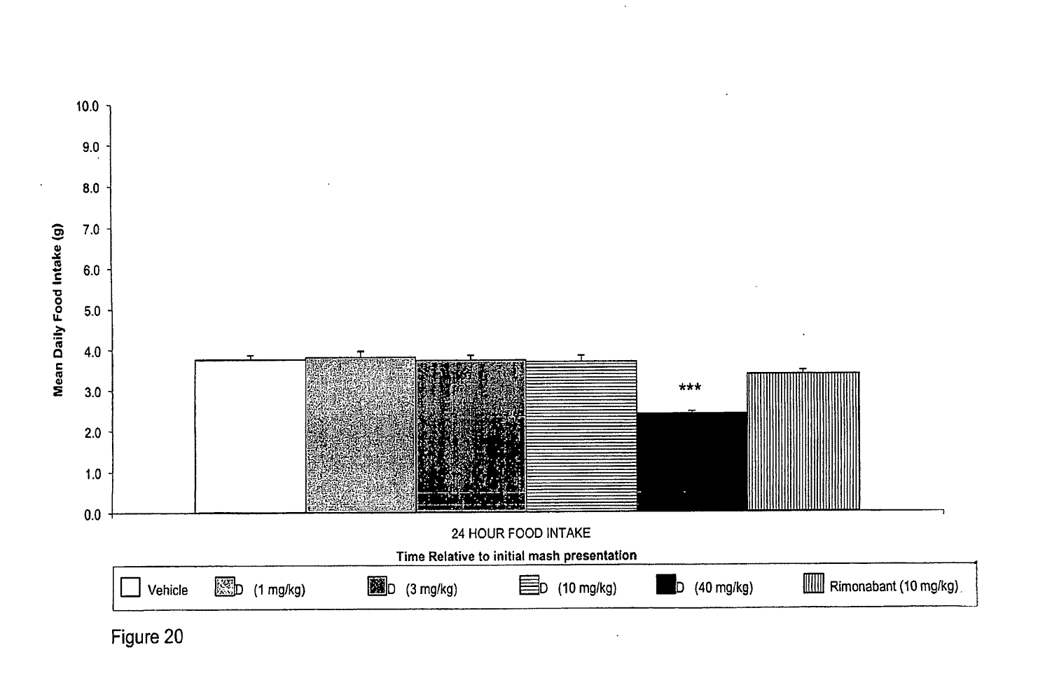

FIG. 20 shows the effect of Treatment D, dosed orally at 1, 3, 10 and 40 mg/kg, and rimonabant, dosed orally at 10 mg/kg, on daily food intake of lean male C57BL/6J mice (n=10). Data was analyzed by one-way ANOVA and Dunnett's test to assess differences versus the control group. *** indicates P<0.001. Treatment D significantly reduced daily food intake when dosed orally at 40 mg/kg. At least a 25% reduction in daily food intake as compared to vehicle was observed when Treatment D was dosed orally at 40 mg/kg.

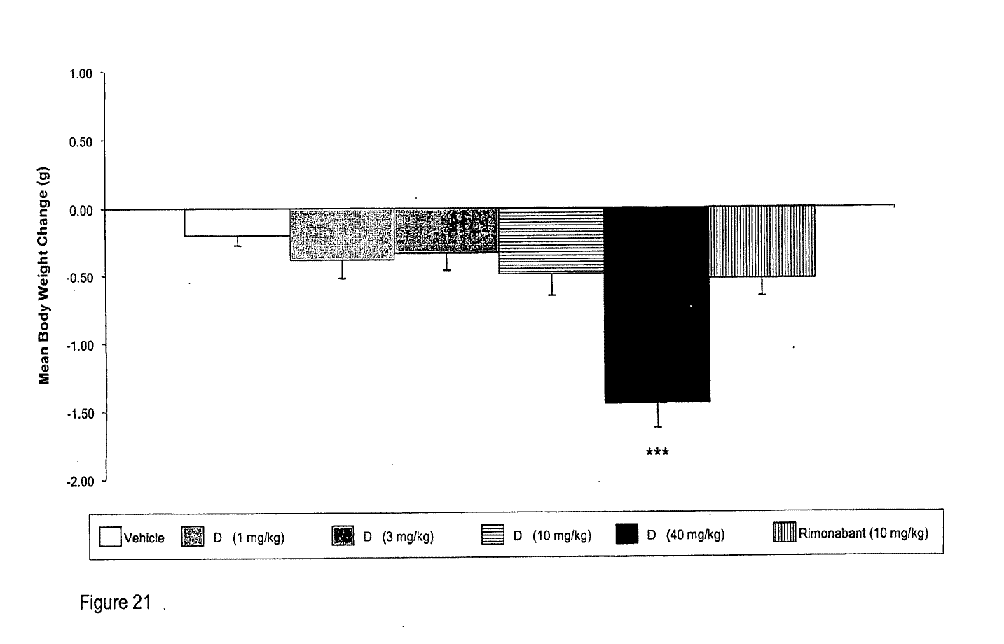

FIG. 21 shows the effect of Treatment D, dosed orally at 1, 3, 10 and 40 mg/kg, and rimonabant, dosed orally at 10 mg/kg, on 24 hour body weight change in lean male C57BL/6J mice (n=10). Data was analyzed by one-way ANOVA and Dunnett's test to assess differences versus the control group. *** indicates P<0.001. Treatment D significantly reduced 24 hour body weight gain when dosed orally at 40 mg/kg. Treatment D, dosed orally at 40 mg/kg, reduced 24 hour body weight gain by at least 300% more than vehicle.

(R)-Norfluoxetine (D)-tartrate alone was dosed per the protocol outlined above and did demonstrate a reduction in weight gain and wet mash intake, though not as much of a reduction as was seen with the combinations of R-norfluoxetine with bupropion.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating obesity, bulimia nervosa, a bulimia-type eating disorder not otherwise specified, anorexia nervosa, metabolic syndrome, or a disorder associated with metabolic syndrome in a mammal, comprising administering to a mammal suffering from obesity, bulimia nervosa, a bulimia-type eating disorder not otherwise specified, anorexia nervosa, metabolic syndrome, or a disorder associated with metabolic syndrome an effective dose of
a compound of any one of formulae 1-6 or a salt thereof, or a solvate of the compound or its salt;
a CB1 antagonist or inverse agonist conjointly with an allosteric potentiator of MC4, an agonist of MC4, an inhibitor of dopamine reuptake, an inhibitor of norepinephrine reuptake, an inhibitor of both dopamine and norepinephrine reuptake, an MAO-B inhibitor, a dopamine D1 agonist, a dopamine D2 agonist, a dopamine D3 agonist, a dopamine D4 agonist, or a dopamine D5 agonist; or
moxonidine or a pharmaceutically acceptable salt thereof conjointly with norfluoxetine enriched for the (R) enantiomer or a pharmaceutically acceptable salt thereof, or a solvate of norfluoxetine enriched for the (R) enantiomer or its salt.

2-4. (canceled)

5. A compound of formula 1:
a compound of formula 3: 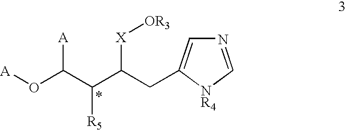
wherein
A independently for each occurrence represents a substituted or unsubstituted aryl or heteroaryl ring;
X represents a substituted or unsubstituted methylene;
R₃ represents H or substituted or unsubstituted C₁₋₆ alkyl, C₁₋₆aralkyl, aryl, heteroaryl, or acyl;
R₄ represents H or substituted or unsubstituted C₁₋₆alkyl and
R₅ represents substituted or unsubstituted C₁₋₆ alkyl acyl C₁₋₆aralkyl, aryl heteroaryl, carbocycle, or heterocycle, provided that when R₅ is substituted or unsubstituted heteroaryl or heterocycle, the atom that is attached to the indicated (*) carbon is a carbon atom;
a compound of formula 4: 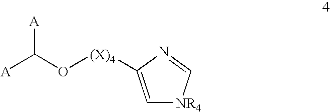
wherein
A independently for each occurrence represents a substituted or unsubstituted aryl or heteroaryl ring;
X independently for each occurrence represents a substituted or unsubstituted methylene; and
R₄ represents H or substituted or unsubstituted C₁₋₆ alkyl; or
a compound of formula 5: 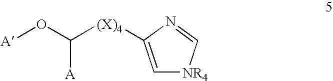
wherein
A represents a substituted or unsubstituted aryl or heteroaryl ring;
A′ represents a substituted aryl or heteroaryl ring;
X independently for each occurrence represents a substituted or unsubstituted methylene; and
R₄ represents H or substituted or unsubstituted C₁₋₆alkyl.

6-9. (canceled)

10. A pharmaceutical composition comprising
a pharmaceutically acceptable carrier, a compound of any one of formulae 1-6 or a salt thereof, or a solvate of the compound or its salt, or norfluoxetine enriched for the (R) enantiomer or a salt thereof, or a solvate of norfluoxetine enriched for the (R) enantiomer or its salt, and at least a second compound selected from: an agonist of MC4; an allosteric potentiator of MC4; an inhibitor of dopamine reuptake; an inhibitor of norepinephrine reuptake; an inhibitor of both dopamine and norepinephrine reuptake; an MAO-B inhibitor; a dopamine D1 agonist; a dopamine D2 agonist; a dopamine D3 agonist; a dopamine D4 agonist; or a dopamine D5 agonist;
wherein the second compound is optionally selected from bupropion; methylphenidate; sibutramine; sertraline; venlafaxine; atomoxetine; amineptine; benztropine; reboxetine; rasagiline; selegiline; deprenyl; lazabemide; quinpirole; talipexole; sumanirole; bromocriptine; ropinirole; pramipexole; levodopa; amantadine; pergolide; fenoldopam; cabergoline; rotigotine; lysuride; 7-OH DPAT; SKF-38393; apomorphine; or a pharmaceutically acceptable salt, metabolite, or stereoisomer thereof; or
a pharmaceutically acceptable excipient and norfluoxetine enriched for the (R) enantiomer or a pharmaceutically acceptable salt thereof, or a solvate of norfluoxetine enriched for the (R) enantiomer or its salt in a range of 1 mg to 10 mg;
wherein said pharmaceutical composition is optionally characterized by one or more of the following:
the pharmaceutically acceptable salt of norfluoxetine enriched for the (R) enantiomer is (R)-norfluoxetine (D)-tartrate; or
the norfluoxetine is substantially free of (S)-norfluoxetine.

11-12. (canceled)

13. The method of claim 1 characterized by one or more of the following:
the CB1 antagonist or inverse agonist is norfluoxetine enriched for the (R) enantiomer or a pharmaceutically acceptable salt thereof, or a solvate of norfluoxetine enriched for the (R) enantiomer or its salt;
the pharmaceutically acceptable salt of norfluoxetine enriched for the (R) enantiomer is optionally (R)-norfluoxetine (D)-tartrate;
the norfluoxetine enriched for the (R) enantiomer is optionally substantially free of (S)-norfluoxetine;
the CB1 antagonist or inverse agonist is administered conjointly with methylphenidate, sibutramine, sertraline, venlafaxine, atomoxetine, amineptine, benztropine, reboxetine, rasagiline, selegiline, deprenyl, lazabemide, quinpirole, talipexole, sumanirole, bromocriptine, ropinirole, pramipexole, levodopa, amantadine, pergolide, fenoldopam, cabergoline, rotigotine, lysuride, 7-OH DPAT, SKF-38393, apomorphine, or a pharmaceutically acceptable salt, metabolite or stereoisomer thereof;
the method is a method of treating metabolic syndrome, a disorder associated with metabolic syndrome, bulimia nervosa, a bulimia-type eating disorder not otherwise specified, or anorexia nervosa comprising administering to a mammal suffering from metabolic syndrome, a disorder associated with metabolic syndrome, bulimia nervosa, a bulimia-type eating disorder not otherwise specified, or anorexia nervosa the CB1 antagonist or inverse agonist conjointly with bupropion or a pharmaceutically acceptable salt thereof, or a metabolite or stereoisomer of bupropion or its salt; and
wherein the disorder associated with metabolic syndrome is optionally selected from diabetes, hypertension, or hyperlipidemia;
the method is a method of treating obesity comprising administering to a mammal suffering from obesity bupropion or a pharmaceutically acceptable salt thereof, or a metabolite or stereoisomer of bupropion or its salt conjointly with norfluoxetine enriched for the (R) enantiomer or a pharmaceutically acceptable salt thereof, or a solvate of norfluoxetine enriched for the (R) enantiomer or its salt,
the norfluoxetine enriched for the (R) enantiomer or a pharmaceutically acceptable salt thereof, or solvate of norfluoxetine enriched for the (R) enantiomer or its salt and bupropion or a pharmaceutically acceptable salt thereof, or a metabolite or stereoisomer of bupropion or its salt are administered in a molar ratio in the range of 1:1 to 20:1, respectively;
when the method comprises administering a) bupropion or a pharmaceutically acceptable salt thereof, or a metabolite or stereoisomer of bupropion or its salt conjointly with b) norfluoxetine enriched for the (R) enantiomer or a pharmaceutically acceptable salt thereof, or a solvate of norfluoxetine enriched for the (R) enantiomer or its salt, the method optionally further comprises administering c) moxonidine or a pharmaceutically acceptable salt thereof;
the compound of any of formulae 1-6 or a salt thereof, or a solvate of the compound or its salt is administered conjointly with an allosteric potentiator of MC4, an agonist of MC4, an inhibitor of dopamine reuptake, an inhibitor of norepinephrine reuptake, an inhibitor of both dopamine and norepinephrine reuptake, MAO-B inhibitor, a dopamine D1 agonist, a dopamine D2 agonist, a dopamine D3 agonist, a dopamine D4 agonist, or a dopamine D5 agonist;
the compound of any of formulae 1-6 or a salt thereof, or a solvate of the compound or its salt is administered conjointly with bupropion, methylphenidate, sibutramine, sertraline, venlafaxine, atomoxetine, amineptine, benztropine, reboxetine, rasagiline, selegiline, deprenyl, lazabemide, quinpirole, talipexole, sumanirole, bromocriptine, ropinirole, pramipexole, levodopa, amantadine, pergolide, fenoldopam, cabergoline, rotigotine, lysuride, 7-OH DPAT, SKF-38393, apomorphine, or a pharmaceutically acceptable salt, metabolite, or stereoisomer thereof; or
said mammal is a human.

14-28. (canceled)

29. A method of
treating obesity, bulimia nervosa, a bulimia-type eating disorder not otherwise specified, or prostate cancer in a mammal, comprising administering to a mammal suffering from obesity, bulimia nervosa, a bulimia-type eating disorder not otherwise specified, or prostate cancer an effective dose of norfluoxetine enriched for the (R) enantiomer, wherein said effective dose is in the range of 1 mg/day to 60 mg/day;
treating anorexia nervosa in a mammal, comprising administering to a mammal suffering from anorexia nervosa an effective dose of norfluoxetine, norfluoxetine enriched for the (R) enantiomer, or a pharmaceutically acceptable salt thereof, or a solvate of norfluoxetine, norfluoxetine enriched for the (R) enantiomer, or its salt;
treating obesity, bulimia nervosa, a bulimia-type eating disorder not otherwise specified or anorexia nervosa in a mammal, comprising administering to a mammal suffering from obesity, bulimia nervosa, a bulimia-type eating disorder not otherwise specified or anorexia nervosa an effective dose of norfluoxetine or a pharmaceutically acceptable salt thereof, or a solvate of norfluoxetine or its salt wherein said effective dose is in the range of 1 mg/day to 10 mg/day, or
treating obesity in a patient being treated with one or more anti-psychotic agents, comprising administering to said patient a CB1 antagonist or inverse agonist;
wherein said method is optionally characterized by one or more of the following:
the one or more anti-psychotic agents are selected from clozapine, olanzapine, quetiapine, risperidone, ziprasidone, aripiprazole, trifluoperazine, flupenthixol, loxapine, perphenazine, chlorpromazine, haloperidol, fluphenazine decanoate, thioridazine, or a pharmaceutically acceptable salt thereof; or
the CB1 antagonist or inverse agonist is norfluoxetine enriched for the (R) enantiomer or a pharmaceutically acceptable salt thereof, or a solvate of norfluoxetine enriched for the (R) enantiomer or its salt;
wherein the norfluoxetine enriched for the (R) enantiomer or a pharmaceutically acceptable salt thereof, or a solvate of norfluoxetine enriched for the (R) enantiomer or its salt is optionally further characterized by one or more of the following:
the pharmaceutically acceptable salt of norfluoxetine enriched for the (R) enantiomer is (R)-norfluoxetine (D)-tartrate; or
the norfluoxetine enriched for the (R) enantiomer is substantially free of (S)-norfluoxetine.

30-39. (canceled)

40. A kit comprising
a. one or more single dosage forms, each comprising a dose of norfluoxetine enriched for the (R) enantiomer or a pharmaceutically acceptable salt thereof, or a solvate or norfluoxetine enriched for the (R) enantiomer or its salt in the range of 1 mg to 60 mg and a pharmaceutically acceptable excipient; and
b. instructions for administering the single dosage forms for the treatment of obesity, anorexia nervosa, bulimia nervosa, or a bulimia-type eating disorder not otherwise specified.

41-80. (canceled)
